(19) United States
(12) Patent Application Publication
(10) Pub. No.: US 2002/0187470 A1
(43) Pub. Date: Dec. 12, 2002

(54) Detection of single nucleotide polymorphisms

(76) Inventors: Warren Michael Casey, Knightdale, NC (US); Jingwen Chen, Morrisville, NC (US); Heidi M. Colton, Apex, NC (US); David Taylor, Hillsborough, NC (US); Michael Phillip Weiner, Cary, NC (US)

Correspondence Address: NEEDLE & ROSENBERG P C, 127 PEACHTREE STREET N E, ATLANTA, GA, 30303-1811, US

(21) Appl. No.: 09/745,317

(22) Filed: Dec. 21, 2000

(51) Int. Cl. C12Q 1/68

(52) U.S. Cl. 435/6

(57) ABSTRACT

Provided is a method of identifying a selected nucleotide in a first nucleic acid utilizing a mobile solid support, as well as a novel read-out method for improving the use of mobile solid support-based read-out technologies for detection of nucleic acid polymorphisms in a target nucleic acid.

### RELATED APPLICATIONS

The present application claims priority from provisional application 60/173,268 filed Dec. 28, 1999. The present application is also a continuation-in-part of PCT/US99/13928 filed Jun. 22, 1999, which claims the benefit of provisional application 60/108,018 filed Nov. 12, 1998, provisional application 60/100,703 filed Sep. 17, 1998, provisional application 60/090,720 filed Jun. 26, 1998 and provisional application 60/090,503 filed Jun. 24, 1998.

### BACKGROUND

1. Field of the Invention

The present invention provides methods for rapid detection of single nucleotide polymorphisms (SNPs) in a nucleic acid sample. The present invention further provides a novel read-out method for improving the use of mobile solid support-based read-out technologies for detection of nucleic acid polymorphisms in a target nucleic acid. The methods can be utilized to detect SNPs in genomic DNA as well as amplified DNA, RNA, etc., thus making them useful for a variety of purposes, including genotyping (such as for disease mutation detection and for parentage determinations in humans and other animals), pathogen detection and identification, and differential gene expression. The present invention further provides a method for identifying a nucleic acid utilizing a run-off sequencing reaction of a relatively short portion of the nucleic acid. The method can be utilized, for example, to identify an EST from only a small portion of the EST and in an analysis of nucleotide polymorphisms. The reactions can be multiplexed to increase data readout capacity.

2. Background Art

Methods of detecting single base polymorphisms have typically involved hybridization reactions. For example, the method of performing a Luminex FlowMetrix-based SNP analysis involves differential hybridization of a PCR product to two differently-colored FACS-analyzable beads. The FlowMetrix system currently consists of uniformly-sized 5 micron polystyrene-divinylbenzene beads stained in eight concentrations of two dyes (orange and red). The matrix of the two dyes in eight concentrations allows for 64 differently-colored beads (8²) that can each be differentiated by a FACScalibur suitably modified with the Luminex PC computer board. In the Luminex SNP analysis, covalently-linked to a bead is a short (approximately 18-20 bases) “target” oligodeoxynucleotide (oligo). The nucleotide positioned at the center of the target oligo encodes the polymorphic base. A pair of beads are synthesized; each bead of the pair has attached to it one of the polymorphic oligonucleotides. A PCR of the region of DNA surrounding the to-be analyzed SNP is performed to generate a PCR product. Conditions are established that allow hybridization of the PCR product preferentially to the bead on which is encoded the precise complement. In one format (“without competitor”), the PCR product itself incorporates a flourescein dye and it is the gain of the flourescein stain on the bead, as measured during the FACScalibur run, that indicates hybridization. In a second format (“with competitor,”) the beads are hybridized with a competitor to the PCR product. The competitor itself in this case is labeled with flourescein. And it is the loss of the flourescein by displacement by unlabeled PCR product that indicates successful hybridization. It has been stated that “with competitor” is more discriminating in SNP analysis.

A method for typing single nucleotide polymorphisms in DNA, labeled Genetic Bit Analysis (GBA) has been described [Genetic Bit Analysis: a solid phase method for typing single nucleotide polymorphisms. Nikiforov T T; Rendle R B; Goelet P; Rogers Y H; Kotewicz M L; Anderson S; Trainor G L; Knapp M R. NUCLEIC ACIDS RESEARCH, (1994) 22 (20) 4167-75]. In this method, specific fragments of genomic DNA containing the polymorphic site(s) are first amplified by the polymerase chain reaction (PCR) using one regular and one phosphorothioate-modified primer. The double-stranded PCR product is rendered single-stranded by treatment with the enzyme T7 gene 6 exonuclease, and captured onto individual wells of a 96 well polystyrene plate by hybridization to an immobilized oligonucleotide primer. This primer is designed to hybridize to the single-stranded target DNA immediately adjacent from the polymorphic site of interest. Using the Klenow fragment of E. coli DNA polymerase I or the modified T7 DNA polymerase (Sequenase), the 3′ end of the capture oligonucleotide is extended by one base using a mixture of one biotin-labeled, one fluorescein-labeled, and two unlabeled dideoxynucleoside triphosphates. Antibody conjugates of alkaline phosphatase and horseradish peroxidase are then used to determine the nature of the extended base in an ELISA format. This paper also describes biochemical features of this method in detail. A semi-automated version of the method, which is called Genetic Bit Analysis (GBA), is being used on a large scale for the parentage verification of thoroughbred horses using a predetermined set of 26 diallelic polymorphisms in the equine genome. Additionally, minisequencing with immobilized primers has been utilized for detection of mutations in PCR products [Minisequencing: A Specific Tool for DNA Analysis and Diagnostics on Oligonucleotide Arrays. Pastinen, T. et al. Genome Research 7:606-614 (1997)].

The effect of phosphorothioate bonds on the hydrolytic activity of the 5′→3′ double-strand-specific T7 gene 6 exonuclease in order to improve upon GBA was studied [The use of phosphorothioate primers and exonuclease hydrolysis for the preparation of single-stranded PCR products and their detection by solid-phase hybridization. Nikiforov T T; Rendle R B; Kotewicz M L; Rogers Y H. PCR METHODS AND APPLICATIONS, (1994) 3 (5) 285-91]. Double-stranded DNA substrates containing one phosphorothioate residue at the 5′ end were found to be hydrolyzed by this enzyme as efficiently as unmodified ones. The enzyme activity was, however, completely inhibited by the presence of four phosphorothioates. On the basis of these results, a method for the conversion of double-stranded PCR products into full-length, single-stranded DNA fragments was developed. In this method, one of the PCR primers contains four phosphorothioates at its 5′ end, and the opposite strand primer is unmodified. Following the amplification, the double-stranded product is treated with T7 gene 6 exonuclease. The phosphorothioated strand is protected from the action of this enzyme, whereas the opposite strand is hydrolyzed. When the phosphorothioated PCR primer is 5′ biotinylated, the single-stranded PCR product can be easily detected colorimetrically after hybridization to an oligonucleotide probe immobilized on a microtiter plate. A simple and efficient method for the immobilization of relatively short oligonucleotides to microtiter plates with a hydrophilic surface in the presence of salt was also described.

DNA analysis based on template hybridization (or hybridization plus enzymatic processing) to an array of surface-bound oligonucleotides is well suited for high density, parallel, low cost and automatable processing [Fluorescence detection applied to non-electrophoretic DNA diagnostics on oligonucleotide arrays. Ives, Jeffrey T.; Rogers, Yu Hui; Bogdanov, Valery L.; Huang, Eric Z.; Boyce-Jacino, Michael; Goelet, Philip L. L. C., Proc. SPIE-Int. Soc. Opt. Eng., 2680 (Ultrasensitive Biochemical Diagnostics), 258-269 (1996)]. Direct fluorescence detection of labeled DNA provides the benefits of linearity, large dynamic range, multianalyte detection, processing simplicity and safe handling at reasonable cost. The Molecular Tool Corporation has applied a proprietary enzymatic method of solid phase genotyping to DNA processing in 96-well plates and glass microscope slides. Detecting the fluor-labeled GBA dideoxynucleotides requires a detection limit of approx. 100 mols/μm². Commercially available plate readers detect about 1000 mols./μm², and an experimental setup with an argon laser and thermoelectrically-cooled CCD can detect approximately 1 order of magnitude less signal. The current limit is due to glass fluorescence. Dideoxynucleotides labeled with fluorescein, eosin, tetramethylrhodamine, Lissamine and Texas Red have been characterized, and photobleaching, quenching and indirect detection with fluorogenic substrates have been investigated.

Although SNP analysis by hybridization is a powerful method, it has several disadvantages. These include; i) a need to synthesize two targets, and possibly two competitor oligonucleotides for each allelic pair, ii) the establishment of the hybridization parameters (buffer content, temperature, time) that will efficiently discriminate between alleles, and iii) an avoidance of regions containing secondary structure that may effect the hybridization parameters.

Current limitations to the GBA methods as described include i) the limited density that can be achieved on a 2-dimensional solid surface, ii) photobleaching, iii) autoflourescence of glass and plastic substrates, iv) difficulty in consistently coupling oligonucleotides to glass, and v) the expense, ease and flexibility of the system for creating new fixed arrays.

The present invention provides a novel system for using a GBA single base chain extension (SBCE) which takes advantage of the powerful matrixing capabilities of a mobile solid support system having multiple dye color/concentration capabilities (e.g., the FlowMetrix system) to overcome the described disadvantages. The present invention further provides a method to improve the detection of reaction products from such polymorphism identification methods. Various detection methods, as described herein and as known in the art, can be enhanced by utilizing the present detection method. Such methods can be combined with the present invention to provide a read out format that is time- and cost-efficient as it provides a means of using any given bead for use, individually, with many primers. This read-out method can be utilized also with many polymorphism detection methods, such as SBCE, OLA and cleavase reaction/signal release (Invader methods, Third Wave Technologies).

### BRIEF DESCRIPTION OF THE DRAWINGS

FIG. 1 shows a 16s rRNA dendrogram for preparation of probes specific for various bacterial contaminants.

FIG. 2 shows a multiplexed genotyping of 7 CEPH DNA samples for 9 SNPs. Oligonucleotide ligation was conducted using PCR-amplified taget nucleic acid from 7 CEPH patients. Biotintylated reporter and avidin-FITC were used.

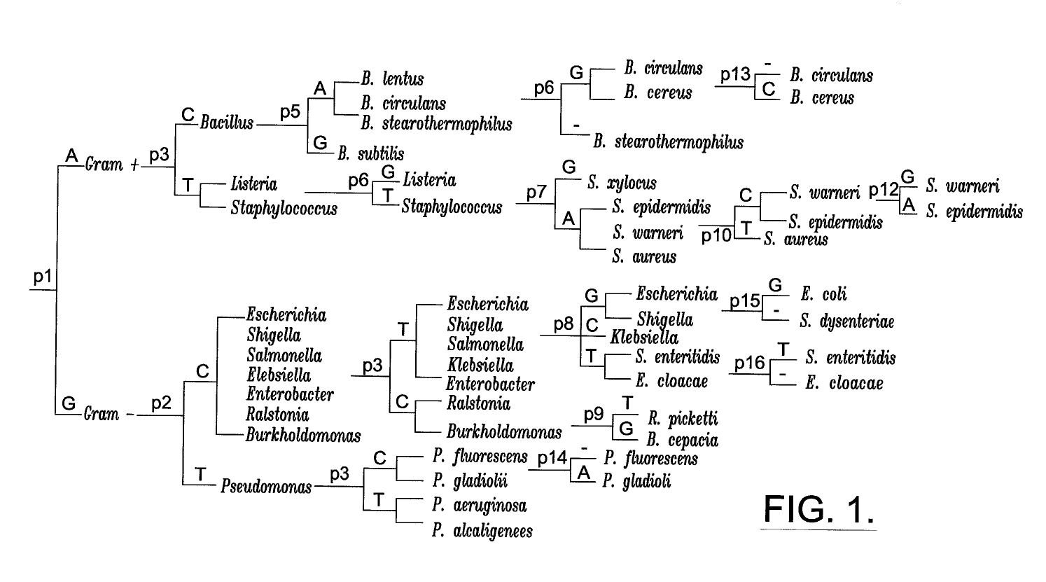

### DETAILED DESCRIPTION

The present invention provides a method whereby a mobile solid support, such as a bead, which is detectably tagged, such as with a dye, a radiolabel, a magnetic tag, or a Quantum Dot® (Quantum Dot Corp.), is utilized in a nucleic acid read out procedure, either a direct readout onto a mobile solid support-linked nucleic acid such as SBCE, OLA or cleavase reaction/signal release (Invader methods, Third Wave Technologies, Madison, Wis.) or an indirect readout (in solution) which is then captured by an intermediate nucleic acid such as by a zipcode attached to a mobile solid support, and the readout product is then analyzed on a selected platform, such as by passing the mobile solid support over a detector (such as a laser detection device) or by passing a detector over the mobile solid support. The intermediate nucleic acid system presents many advantages. For example, in a ligation reaction, ligation of a reporter probe to a target oligonucleotide in solution is more efficient and reproducible than ligation to target oligonucleotides that have been directly coupled to microspheres. In addition, ZipCodes allow the use and reuse of a defined set of optimally coupled microspheres. One cZipCode-coupled microsphere type may be used to analyze new single nucleotide polymorphisms instead of preparing new microspheres each time a new single nucleotide polymorphisms arises. The intermediate nucleic acid system system may also be applied to protein-based systems (for example, anti-cytokine antibody may be coupled to a ZipCode oligo for cytokine analysis).

The present invention provides a novel system for SNP readout using an encoded mobile solid support which takes advantage of the powerful matrixing capabilities of a mobile solid support system. In one embodiment, the system uses a GBA single base chain extension (SBCE). In another embodiment, the system utilizes an oligonucleotide ligation assay. In yet another embodiment, the system uses an enzymatic or chemical read-out method whereby an enzyme or chemical is used to modify or endonucleolytically cleave a mismatched base at the polymorphic site, resulting in the loss of an attached reporter or said modification resulting in a labeling means for the identification of the modification. Thus, in a further embodiment, the system utilizes an endonuclease cleavase/signal release method (Invader methods, Third Wave Technologies) (see, e.g., Marshall et al. J. Clin. Microbiol. 35(12):3156-3162 (1997); Brow, et al. J. Clin. Microbiol. 34(12):3129-3137 (1997)). In another embodiment, fluorescence energy transfer (FET) is used with fluorescence quenching as a readout.

In the cleavase enzyme readout, target nucleic acid (e.g., PCR product or genomic DNA) hybridizes to both a complementary Invader probe and a Signal probe; a cleavase enzyme recognizes the specific structure formed between the target nucleic acid, Invader probe, and Signal probe, and cleaves the Signal probe at the branch site and thereby releases the signal for detection. Another Signal probe can then bind to the nucleic acid and the cleavase reaction begins anew. This process is repeated many times and thereby increases the signal amplification. The essence for cleavase to work is the presence of an overlapping base of the Invader probe with the signal base. In an improved version, named Invader Squared, two rounds of Invader are performed simultaneously. The primary invader reaction involves using SNP-specific target DNA, the resulting cleavase-product becomes functional in a secondary Invader reaction with a universal signal probe and universal complementary target DNA. After the second round invader assay, a linear signal amplification of greater than 10⁶ signal/target/hr is obtained.

The present invention further provides a novel read-out method for improving the use of mobile solid support-based read-out technologies for detection of nucleic acid polymorphisms in a target nucleic acid utilizing a target oligonucleotide having a first complementarity region complementary to the target nucleic acid and a second complementarity region, 5′ to the first complementarity region, complementary to a capture oligonucleotide, which capture oligonucleotide is linked to a mobile solid support. The improved method can be applied to any of several methods of identifying a nucleic acid polymorphism, such as oligonucleotide ligation assay (OLA) or single base chain extension (SBCE), as described herein. The methods can be utilized to detect SNPs in genomic DNA as well as amplified DNA, RNA, etc., thus making it useful for a variety of purposes, including genotyping (such as for disease mutation detection and for parentage determinations in humans and other animals), pathogen detection and identification, and differential gene expression.

The present invention further provides the development of a simple method for multiplexing short sequencing reads (about 16 bases) in the same lane. One application to which this method can be applied is high-throughput yeast two-hybrid analysis. In this analysis, it is desired to sequence short regions of the interacting proteins, and then use a large database to determine the hit identification. Because each bait analyzed generates approximately 100 hits, the present method to increase the efficiency of analysis was needed and therefore developed.

The invention can be utilized to analyze a nucleic acid sample that comprises genomic DNA, amplified DNA, such as a PCR product, cDNA, cRNA, a restriction fragment or any other desired nucleic acid sample. When one performs one of the herein described methods on genomic DNA, typically the genomic DNA will be treated in a manner to reduce viscosity of the DNA and allow better contact of a primer or probe with the target region of the genomic DNA. Such reduction in viscosity can be achieved by any desired method, which are known to the skilled artisan, such as DNase treatment or shearing of the genomic DNA, preferably lightly. Amplified DNA can be obtained by any of several known methods. Sources of genomic DNA are numerous and depend upon the purpose of performing the methods, but include any tissue, organ or cell of choice. Oligonucleotides can be generated by amplification or by de novo synthesis, for example. Complementary nucleic acids, i.e., cRNA (obtained from a process wherein DNA is primed with a T7-RNA polymerase/specific sequence primer fusion, then T7 RNA polymerase is added to amplify the first strand to create cRNA) and cDNA, can be obtained by standard methods known in the art.

Thus, in the present methods, “nucleic acid” includes any of, for example, an oligonucleotide, genomic DNA, a 16s ribosomal RNA, a PCR product, a DNA fragment, an RNA molecule, a cDNA molecule or a cRNA molecule, the nucleic acid primer is an oligonucleotide, a PCR product, LCR (ligase chain reaction) product, a DNA fragment, an RNA molecule, a cDNA molecule or a cRNA molecule. Often a primer or a probe in an example is an oligonucleotide, but the source of the primers or probes is not so limited herein.

As used in the claims, “a” and “an” can mean one or more, depending upon the context in which it is used.

In the basic SBCE method, a single oligonucleotide is attached to a detectably tagged, mobile solid support, such as a bead or a rod, preferably that can be processed for detection of the tag quickly once the desired reaction has taken place, such as by a FACS-type system. For example, if one will ultimately fix the support in place prior to detection, a “tentagel” (“octopus”) can be used, then fixed in place prior to detection. Any desired tag can be utilized, such as a fluorescent tag, a radiolabel, or a magnetic tag. Other detection systems can be used, preferably, however, wherein the mobile solid support is passed over a detection device, such as a laser detection device, capable of detecting and discerning the selected tags and labels (see, e.g., PCT publication WO 9714028). Detection systems can also be utilized wherein the mobile solid support, after performing any reactions, is fixed onto a two-dimensional surface and a detection device, such as a laser detection device, is passed over the fixed mobile solid support. The mobile solid support can comprise any useful material, such as polystyrene-divinylbenzene. Detection of the mobile solid support and any nucleic acid or nucleotide associated with it, can be performed by FACS-based method, such as the Luminex FlowMetrix™ system.

In a typical assay, the oligonucleotide is designed such that the 5′ end is coupled to the bead. The 3′ base ends at a nucleotide chosen relative to the polymorphic base, depending upon the assay being performed. For example, the 3′ base of this primer or probe can end at the nucleotide 5′ to the polymorphic base, it can end with a base corresponding to the polymorphic base. The length of the oligo in the SBCE method is not critical, but it does need to be long enough to support hybridization by a nucleic acid sample, such as a PCR product generated from a region surrounding the SNP. Depending upon the assay to be performed, the primer or probe can be designed wherein an exact match is required or it may be designed to allow some mismatch upon initial hybridization to the sample nucleic acid.

In a typical assay, a nucleotide capable of chain termination is utilized. Such chain termination is a termination event that occurs before the same labeled base occurs again in the target sequence. Such nucleotides are known in the art and include, for example, a dideoxynucleotide (when polymerase is used in the extension reaction), a thiol derivative (when polymerase is used in the extension reaction), a 3′ deoxynucleotide (using reverse transcriptase in the extension reaction), or a 3′ deoxyribonucleotide (using reverse transcriptase in the extension reaction). Any of these nucleotides can be, for example, a dinucleotide, a trinucleotide, or a longer nucleic acid. Thus, one can have, for example, a bank of dinucleotides or longer nucleic acids such that within the bank one has optional nucleotides at more than one location.

Thus, in the present method, the labeling step is typically performed in solution (thus providing efficient hybridization), and the analysis step can be performed either in solution or on a solid, non-mobile support.

The present invention therefore provides a method of identifying a selected nucleotide in a first nucleic acid comprising

(a) contacting the first nucleic acid with a nucleic acid primer linked at its 5′ end to a detectably tagged mobile solid support wherein the nucleic acid primer comprises a region complementary to a section of the first nucleic acid that is directly 3′ of and adjacent to the selected nucleotide, under hybridization conditions that allow the first nucleic acid and the nucleic acid primer to form a hybridization product;

(b) performing a primer extension reaction with the hybridization product and a detectably labeled, identified chain-terminating nucleotide under conditions for primer extension; and

(c) detecting the presence or absence of a label incorporated into the hybridization product,

the presence of a label indicating the incorporation of the labeled nucleotide into the hybridization product, and the identity of the incorporated labeled nucleotide indicating the identity of the nucleotide complementary to the selected nucleotide, thus identifying the selected nucleotide in the first nucleic acid.

In a specific embodiment, a primer is designed such that its 3′ base ends at the nucleotide immediately 5′ of the polymorphic base. A set of 4 dideoxynucleotide triphosphate mixtures are generated. Each mixture contains one of four labeled dideoxynucleotide molecules that have been chemically-coupled to a flourescein molecule (i.e., ddATP-F, ddCTP-F, ddGTP-F or ddTTP-F), and three non-labeled dideoxynucleotide triphosphates. In one format, the PCR product is added to the bead and the bead aliquoted into 2 or more tubes. The chain-terminating mixtures are dispensed to the tubes and a polymerase is added to generate the SBCE reaction tubes. The polymerase will extend a base onto the 3′ end of the bead-attached oligo, this base being the complement of the base at the polymorphic site. The reaction tubes are analyzed by FlowMetrix and the appearance of a label in a particular reaction tube on a particular bead will indicate the polymorphic base at the site.

A comparison of the present method with a hybridization method is illustrative of the utility of the present invention. In the SNP analysis by hybridization, 2 oligos on 2 beads in the same tube are used to generate the material to be read for analysis. In the SCBE method, the same oligo on the same bead is analyzed in 2 tubes with 2 different labeled dideoxynucleotides. Although the method has been exemplified herein using flourescein as the dye read-out, one can couple this method with biotinylated or other appropriately-modified nucleotides.

The present methods can be performed wherein the chain terminating nucleotide is a dideoxynucleotide and the primer extension is performed in the presence of one labeled, identified dideoxynucleotide and three different, non-labeled dideoxynucleotides. In another embodiment, the chain terminating nucleotide is a dideoxynucleotide, wherein the primer extension is performed in the presence of a first identified dideoxynucleotide labeled with a first detectable label, a second identified dideoxynucleotide labeled with a second detectable label, a third identified dideoxynucleotide labeled with a third detectable label and a fourth identified dideoxynucleotide labeled with a fourth detectable label, and wherein detection of the presence of the first, the second, the third or the fourth detectable label in the hybridization product indicates the identity of the nucleotide complementary to the selected nucleotide as the first, the second, the third or the fourth dideoxynucleotide, respectively.

It is possible to thermal cycle the FlowMetrix beads. Thus, one can perform a genomic scan using the SBCE method. In this method, the genomic DNA could be sheared, or treated with DNase to reduce viscosity, and cycled against oligos attached to the beads. Because of the vastly greater complexity of the template DNA, it may necessitate the need for extended hybridization optimization and cycling times. Since one would be essentially performing a Cot analysis on the beads. Use of these beads and SBCE for SNP identification and DNA sequencing should be apparent from the above description.

Thus, the present invention provides a method of determining a selected nucleotide polymorphism in genomic DNA treated to reduce viscosity comprising

(a) performing an amplification of the genomic DNA using a first nucleic acid primer comprising a region complementary to a section of one strand of the nucleic acid that is 5′ of the selected nucleotide, and a second nucleic acid primer complimentary to a section of the opposite strand of the nucleic acid downstream of the selected nucleotide, under conditions for specific amplification of the region of the selected nucleotide between the two primers, to form a PCR product;

(b) contacting the PCR product with a first nucleic acid linked at its 5′ end to a detectably tagged mobile solid support, wherein the first nucleic acid comprises a region complementary to a section of one strand of the PCR product that is directly 5′ of and adjacent to the selected nucleotide, under hybridization conditions to form a hybridization product;

(c) performing a primer extension reaction with the hybridization product and a detectably labeled, identified chain-terminating nucleotide under conditions for primer extension;

(d) detecting the presence or absence of a label incorporated into the hybridization product, the presence of a label indicating the incorporation of the labeled chain-terminating nucleotide into the hybridization product, and the identity of the incorporated labeled chain-terminating nucleotide indicating the identity of the nucleotide complementary to the selected nucleotide; and

(e) comparing the identity of the selected nucleotide with a non-polymorphic nucleotide,

a different identity of the selected nucleotide from that of the non-polymorphic nucleotide indicating a polymorphism of that selected nucleotide. The PCR product can be in single-stranded form.

The present invention further provides a method of determining a selected nucleotide polymorphism in genomic DNA treated to reduce viscosity comprising

(a) performing an amplification of the genomic DNA using as a primer an oligonucleotide comprising a first region having a T7 RNA polymerase promoter and a second region complementary to a section of one strand of the nucleic acid that is directly 5′ of the selected nucleotide, and using T7 RNA polymerase to amplify one strand into cRNA and using reverse transcriptase to amplify the second strand complementary to the cRNA strand, under conditions for specific amplification of the region of the nucleotide between the two primers, to form an amplification product;

(b) contacting the amplification product with a first oligonucleotide linked at its 5′ end to a detectably tagged mobile solid support under hybridization conditions to form a hybridization product;

(c) performing a primer extension reaction with the hybridization product and a detectably labeled, identified chain-terminating nucleotide under conditions for primer extension;

(d) detecting the presence or absence of a label incorporated into the hybridization product, the presence of a label indicating the incorporation of the labeled chain-terminating nucleotide into the hybridization product, and the identity of the incorporated labeled chain-terminating nucleotide indicating the identity of the nucleotide complementary to the selected nucleotide; and

(e) comparing the identity of the selected nucleotide with a non-polymorphic nucleotide,

a different identity of the selected nucleotide from that of the non-polymorphic nucleotide indicating a polymorphism of that selected nucleotide.

The labeled chain-terminating nucleotide can be, for example, a 3′deoxynucleotide, a 3′deoxyribonucleotide, a thiol nucleotide derivative or a dideoxynucleotide. The amplification product can be in single-stranded form.

Furthermore, one can design and synthesize some primers to sit just downstream of the nucleic acids attached to the beads. These can be the primers used to i) make the first strand cDNA, and, ii) with a set that has attached to it the T7 RNA polymerase, can be used to make cRNA. To make the second strand, if needed for the cRNA, one can use a second primer set that sits outside of the sequence attached to the beads, but just upstream of it. By having the primers off the bead-oligo, they shouldn't interfere by binding. The primers can be made FITC-labeled for the cDNA.

The present method further provides a method of determining a selected nucleotide polymorphism in genomic DNA treated to reduce viscosity comprising

(a) contacting the genomic DNA with a first primer linked at its 5′ end to a detectably tagged mobile solid support, wherein the first primer comprises a first region complementary to a section of one strand of the genomic DNA that is directly 5′ of and adjacent to the selected nucleotide under hybridization conditions for forming a specific hybridization product;

(b) performing a primer extension reaction with the specific hybridization product and a detectably labeled, identified chain-terminating nucleotide under conditions for primer extension;

(c) detecting the presence or absence of a label incorporated into the hybridization product, the presence of a label indicating the incorporation of the labeled chain-terminating nucleotide into the hybridization product, and the identity of the incorporated labeled chain-terminating nucleotide indicating the identity of the nucleotide complementary to the selected nucleotide; and

(d) comparing the identity of the selected nucleotide with a non-polymorphic nucleotide,

a different identity of the selected nucleotide from that of the non-polymorphic nucleotide indicating a polymorphism of that selected nucleotide.

The DNA can be in single-stranded form. The labeled chain-terminating nucleotide can be, for example, a 3′deoxynucleotide, a 3′deoxyribonucleotide, a thiol nucleotide derivative or a dideoxynucleotide. In such a method, the hybridization time should be of a length sufficient to allow hybridization of the first primer to the genomic DNA since the genomic DNA has not been amplified in this specific embodiment. Thus relatively long hybridization times may be utilized, such as, for example, 12 hours, 24 hours, 48 hours, as is known in the art for hybridization to genomic DNA (see, e.g., Sambrook, et al. Molecular Cloning: A Laboratory Manual, 2d ed. Cold Spring Harbor Laboratory, Cold Spring Harbor, N.Y. 1989).

For any of the herein described reactions, alternative polymerases can be employed, such as a polymerase with a temperature condition for function, or a polymerase with a particular specificity for nucleotides, such as a polymerase that preferentially incorporates dideoxynucleotides (see, e.g., Sambrook, et al.). The skilled artisan is familiar with such polymerases, and new polymerases, as they are discovered, can be incorporated into the methods, given the teachings herein.

The present invention additionally provides the use of the beads in an oligonucleotide-ligation assay (OLA) format, i.e., in which one can hybridize genomic DNA, cRNA or PCR product to a first nucleic acid attached to the bead, then come in with a second nucleic acid with a fluorescent label, then add ligase, and wherein the second nucleic acid has at its 3′ end the polymorphic bases. Thus, the present invention provides a method of identifying a selected nucleotide in a first nucleic acid comprising

(a) contacting the first nucleic acid with (i) a second nucleic acid linked at its 5′ end to a detectably tagged mobile solid support wherein the second nucleic acid comprises a region complementary to a section of the first nucleic acid that is directly 3′ of the selected nucleotide and wherein the second nucleic acid terminates at its 3′ end in a test nucleotide positioned to base-pair with the selected nucleotide, and (ii) a third, fluorescently labeled nucleic acid, wherein the third nucleic acid comprises a region complementary to a section of the second nucleic acid that is directly 5′ of and adjacent to the selected nucleotide, under hybridization conditions that allow the first nucleic acid and the second nucleic acid to form a hybridization product and the first nucleic acid and the third nucleic acid to form a hybridization product;

(b) adding to the hybridization product a ligase under ligation conditions; and

(c) detecting the presence or absence of the fluorescent label, after dissociation of the hybridized nucleic acids, in the nucleic acid linked to the mobile solid support,

the presence of the label indicating the ligation of the labeled third nucleic acid to the second nucleic acid linked to the mobile solid support, and the identity of the test nucleotide in the second nucleic acid indicating the identity of the nucleotide complementary to the selected nucleotide, thus identifying the selected nucleotide. This oligonucleotide ligation assay can be performed both (a) wherein the polymorphic base is located at the 5′ side of either the reporter or acceptor oligonucleotide, or (b) wherein the polymorphic base is located at the 3′ side of either the reporter or acceptor oligonucleotide.

The first nucleic acid can be genomic DNA (treated to reduce viscosity, e.g., by DNase treatment or by shearing), amplified nucleic acid such as a PCR product, an oligonucleotide, a 16s ribosomal RNA, a DNA fragment, an RNA molecule, a cDNA molecule, a cRNA molecule, restriction enzyme-generated DNA fragment, size-selected DNA, Bridge-amplified DNA, 16S RNA, 16S DNA or any other desired nucleic acid. Any selected ligase can be used, such as T4 DNA ligase. A thermostable ligase would be particularly useful. See, generally Wu and Wallace, Genomics 4: 560-569 (1989).

The present invention additionally encompasses the use in the OLA readout of degenerate reporter oligonucleotides, preferably the use of 8-mer oligonucleotides wherein 6 of the bases are chosen to be specific to the target nucleic acid and 2 of the bases are variable, or wobble or degenerate, positions. The degeneracy can be placed in any position in the reporter oligonucleotide; however, preferable positions can be positions 3, 4, 5, and 6. Preferable variable position combinations in a selected oligonucleotide can be positions 3 and 6, positions 4 and 5, and positions 3 and 4. Thus, one can synthesize all possible “6+2-mers” as reagents for use in an assay, whereas synthesis of all possible 8-mers is not practicable. Furthermore, non-natural derivatives, such as inosine, can be utilized in the reporter oligonucleotides. For example, the present invention includes an OLA readout wherein the reporter oligonucleotide is an 8-base complementary 8-mer conjugated to a reporter molecule or hapten to which a reporter molecule can be conjugated by means of a hapten-recognizing intermediary (e.g., antibody, avidin, streptavidin). The present invention further includes an OLA readout wherein the reporter oligonucleotide is an 6-base complementary 8-mer (“6+2-mer”) conjugated to a reporter molecule or hapten to which a reporter molecule can be conjugated by means of a hapten-recognizing intermediary. The two non-complementary bases can be any of the four natural bases or can be a non-natural derivative capable of forming a non-helix disturbing duplex structure. The non-complementary bases can preferably be located at positions 3 and 6 or positions 4 and 5. Non-natural base derivatives and/or 6+2-mers can be components of a kit for use in performing the detection methods described herein.

Further, one can employ a ‘Taqman’ approach wherein one incorporates Dye quenchers and Dye acceptors into the attached oligos and asks for the polymerase to remove the dye quencher in a repair reaction.

The invention further employs hybridization methods wherein two nucleic acids are hybridized to the sample nucleic acid but the step of ligation can be omitted and a match instead detected by fluorescence energy transfer between the two nucleic acids hybridized to the sample nucleic acid. The two hybridizing nucleic acids are designed such that the 3′ end of the nucleic acid linked to the bead is a test base, and when it is complementary to the polymorphic base, and a single wavelength of light is directed onto the sample, one can detect a transfer of energy, read as a second wavelength of light. A second reader can be employed for this detection of this second wavelength. Thus, the present invention provides a method of identifying a selected nucleotide in a first nucleic acid comprising

(a) contacting the first nucleic acid with

(i) a second nucleic acid linked at its 5′ end to a detectably tagged mobile solid support and linked at its 3′ end to a fluorescent label, wherein the second nucleic acid comprises a region complementary to a section of the first nucleic acid that is directly 3′ of the selected nucleotide and wherein the second nucleic acid terminates at its 3′ end in a test nucleotide positioned to base-pair with the selected nucleotide, and

(ii) a third nucleic acid fluorescently labeled at its 5′ end, wherein the third nucleic acid comprises a region complementary to a section of the second nucleic acid that is directly 5′ of and adjacent to the selected nucleotide,

(b) under hybridization conditions that allow the first nucleic acid and the second nucleic acid to form a hybridization product and the first nucleic acid and the third nucleic acid to form a hybridization product; and

(c) detecting the presence or absence of fluorescent energy transfer between the fluorescent label at the 3′ end of the second nucleic acid and the fluorescent label at the 5′ end of the third nucleic acid, the presence of fluorescent energy transfer indicating the hybridization of the test nucleotide to the first nucleic acid, and the identity of the hybridized test nucleotide in the second nucleic acid indicating the identity of the nucleotide complementary to the selected nucleotide, thus identifying the selected nucleotide. The detection of the fluorescence energy transfer (FET) can be performed after dissociation of the hybridized nucleic acids.

The present invention also provides a method for determining the sequence of a polymorphic base comprising: a first nucleic acid attached at a 5′ end to a mobile solid support and having a 3′ end adjacent to a polymorphic base on a second nucleic acid; a third nucleic acid with an attached reporter moiety that is complementary to a region adjacent to the polymorphic base of the second nucleic acid; the first nucleic acid and the third nucleic acid together defining a gap opposite the polymorphic base; a nucleotide that is complementary to one of a set of two possible polymorphic bases, a polymerase, and a ligase; wherein the polymerase is able to polymerize the nucleotide across the gap if the nucleotide is complementary to the polymorphic base; the ligase is able to ligate the newly polymerized nucleotide to the reporter-attached third nucleic acid; and a means for detecting the reporter covalently linked to the bead. Specifically, the present invention provides a method of identifying a selected nucleotide in a first nucleic acid comprising

(a) contacting the first nucleic acid with

(i) a second nucleic acid linked at its 5′ end to a detectably tagged mobile solid support, wherein the second nucleic acid comprises a region complementary to a section of the first nucleic acid that is directly 3′ of and immediately adjacent to the selected nucleotide, and

(ii) a third nucleic acid fluorescently labeled, wherein the third nucleic acid comprises a region complementary to a section of the second nucleic acid that is directly 5′ of and adjacent to the selected nucleotide,

under hybridization conditions that allow the first nucleic acid and the second nucleic acid to form a hybridization product and the first nucleic acid and the third nucleic acid to form a hybridization product, wherein the first, second and third nucleic acids form a hybridization product that defines a gap opposite the selected nucleotide;

(a) adding a test nucleotide, a polymerase and a ligase, under conditions for polymerization and ligation; and

(c) detecting the presence or absence of the fluorescent label, after dissociation of the hybridized nucleic acids, in the nucleic acid linked to the mobile solid support,

the presence of the label indicating the polymerization of the test nucleic acid to the second nucleic acid and ligation of the labeled third nucleic acid to the second nucleic acid linked to the mobile solid support, and the identity of the test nucleotide indicating the identity of the nucleotide complementary to the selected nucleotide, thus identifying the selected nucleotide.

The polymerase can preferably be a non-strand displacing polymerase. Further, it can be a thermostable polymerase. The ligase can be a DNA ligase. Further, it can be a thermostable ligase.

The present invention further provides a method of detecting a single base polymorphism comprising using an enzyme or chemical to modify or endonucleolytically cleave a mismatched base at the polymorphic site in a nucleic acid, resulting in the loss of an attached reporter or in a modification, and detecting a loss of the reporter or detecting the modification, thus resulting in a labeling means for the identification of the modification. In one example, an end-labeled (such as with FITC) genomic fragment or a labeled (such as with FITC) PCR fragment is hybridized to an oligonucleotide and attached to a bead, then the construct is treated with an enzyme that recognizes and/or restricts mispaired DNA (such as FITC-labeled recA, mutS or T7 enzyme) and analyzed for the addition or loss of the label. In another example, a chemical recognizing single stranded regions of DNA and capable of modifying the region is utilized, and the modification is detected.

Furthermore, any of the herein described methods can be utilized in a method for quantitating expression of a selected nucleic acid in a sample. Thus, it can be used, for example, for differential gene expression wherein the expression of a selected gene is quantitated and compared to a standard or some other reference. For this method, a gene fragment from a region of interest or a region that distinguishes the gene (or allele or haplotype or polymorphism) of interest is linked at its 5′ end to a detectably labeled mobile solid support; message (e.g., RNA, cDNA, cRNA) is hybridized to the fragment, and fluorescence is quantitated by performing a primer extension reaction, a ligase reaction or a hybridization/fluorescence energy transfer reaction, such as that described herein. The nucleic acid probe can comprise a region complementary to a section of the selected nucleic acid unique to the nucleic acid. A standard, such as that from a normal subject, or a diseased/afflicted subject, or a particular tissue or organ, or a particular species, can be used as a comparison reference to draw conclusion regarding the quantity detected in the sample.

Specifically, the present invention provides a method of detecting a result from an identification reaction to identify a selected nucleotide in a target nucleic acid comprising:

a) contacting a target oligonucleotide comprising a first complementarity region and a second complementarity region, wherein the second complementarity region is 5′ of the first complementarity region and wherein the first complementarity region comprises a region complementary to a section of the target nucleic acid that is directly 3′ of and adjacent to the selected nucleotide, with a sample comprising the target nucleic acid, under hybridization conditions that allow the formation of a hybridization product between the first complementarity region of the target oligonucleotide and a region of the target nucleic acid complementary to the first complementarity region of the target oligonucleotide, to form a first hybridization product;

b) performing a selected identification reaction with the first hybridization product to determine the identity of the selected nucleotide wherein a selectively labeled detection product comprising the second complementarity region of the target oligonucleotide can be formed;

c) isolating the detection product by contacting the detection product with a capture oligonucleotide that is covalently coupled to a mobile solid support, wherein the capture oligonucleotide comprises a nucleic acid sequence complementary to the second complementarity region of the target oligonucleotide, under hybridization conditions to form a second hybridization product; and

d) detecting and/or identifying the label of the labeled detection product in the second hybridization product,

the presence and or identity of the label indicating the identity of the selected nucleotide in the target nucleic acid.

The basic method thus involves the use of a capture oligonucleotide, linked to a mobile solid support (such as a bead), to isolate a reaction product from a reaction. To facilitate this isolation, a “target oligonucleotide” is designed which comprises, in addition to a first complementarity region, which is a region complementary to a region of the target nucleic acid, a second complementarity region, which is located 5′ of the first complementarity region, and which is complementary to the nucleotide sequence of the capture oligonucleotide. Thus, before or after a reaction (such as SBCE or OLA), the capture oligonucleotide can be utilized in a hybridization reaction to isolate the target oligonucleotide in its reacted form (e.g., as a ligation product or as a primer extension product). Thus, one is not obligated, as in many other assays, to synthesize a bead specifically for each oligonucleotide (e.g., the “first complementarity region of the target oligonucleotide in the present invention) that is to be hybridized to the target nucleic acid.

The present invention additionally encompasses the use in the OLA readout of degenerate reporter oligonucleotides, preferably the use of 6+2-mers as described herein. Such reporter oligonucleotides can be a component of a useful kit for performing the detection methods herein.

The capture oligonucleotide can be designed such that it does not specifically hybridize, i.e., is not sufficiently complementary for specific hybridization to occur, to the target nucleic acid. For example, it can include nucleotide usage not typically found in the target species (such as human). If the target sequence is fully known, the capture sequence can be selected as a sequence which does not occur in the target sequence. A capture oligonucleotide can be of any desired length so long as it is sufficiently long so as to selectively hybridize to a first complementarity region of a target oligonucleotide (under selective hybridization conditions, e.g., stringent hybridization conditions, as known to one skilled in the art), and not so long as to interfere with either the identification reaction being performed with the target oligonucleotide or the hybridization reaction between the capture oligonucleotide and the target oligonucleotide. The capture oligonucleotide length selected can also be a function of how many different capture oligonucleotides one desires to use in any selected use. For example, the capture oligonucleotide can be 8, 10, 12, 15, 18, 20, 21, 22, 23, 24, 25, 26, 27, 28, 29, 30, 35, 40 or more nucleotides. A preferred length is around 25 nucleotides, such as 23, 24, 25, 26, 27 or 28 nucleotides. However, other oligonucleotide lengths can be utilized. Optimal length for any specific use can be determined according to the specific nucleic acid composition, as will be known to those skilled in the art.

One can advantageously create a bank of several capture oligonucleotides, each linked to a different color of bead. A bank of complementary regions can be maintained for use in generating target oligonucleotides for any specific target nucleic acid. Thus, one can utilize a defined set of beads, and simply create new target nucleotides as necessary for any given detection task.

The present invention provides a method of detecting a reaction product to identify a selected nucleotide in a target nucleic acid comprising:

a) contacting a target oligonucleotide comprising a first complementarity region and a second complementarity region, wherein the first complementarity region comprises the oligonucleotide primer and the second complementarity region comprises a nucleic acid sequence complementary to a capture oligonucleotide, and wherein the oligonucleotide primer comprises a region complementary to a section of the target nucleic acid that is directly 3′ of and adjacent to the selected nucleotide, with a sample comprising the target nucleic acid, under hybridization conditions that allow the formation of a hybridization product between the first complementarity region of the target oligonucleotide and a region of the target nucleic acid complementary to the first complementarity region of the target oligonucleotide, to form a first hybridization product;

b) performing a primer extension reaction with the first hybridization product and a detectably labeled, identified chain-terminating nucleotide under conditions for primer extension to form a primer extension product;

c) isolating the primer extension product by contacting the primer extension product with a capture oligonucleotide that is covalently coupled to a mobile solid support, wherein the capture oligonucleotide comprises a nucleic acid sequence complementary to the second complementarity region of the target oligonucleotide, under hybridization conditions to form an isolated second hybridization product; and

d) detecting the presence or absence of a label in the isolated second hybridization product,

the presence of a label indicating the incorporation of the labeled nucleotide into the primer extension product, and the identity of the identified incorporated labeled nucleotide indicating the identity of the nucleotide complementary to the selected nucleotide, thus identifying the selected nucleotide in the target nucleic acid.

In a typical assay, the target oligonucleotide is designed such that the 5′ end comprises second complementarity region and later allows for hybridization to a complementary capture oligonucleotide linked to a mobile solid support, and the 3′ end comprises a first complementarity region complementary a region of the target nucleic acid just 3′ of the polymorphic base. The 3′ base ends at a nucleotide chosen relative to the polymorphic base, depending upon the assay being performed. For example, the 3′ base of this target oligonucleotide can end at the nucleotide 5′ to the polymorphic base, or it can end with a base corresponding to the polymorphic base. The present invention additionally provides the use of the beads in an oligonucleotide-ligation assay (OLA) format, i.e., in which one can hybridize genomic DNA, cRNA or PCR product to a target oligonucleotide having a first complementarity region that is complementary to a section of the target nucleic acid that is directly 3′ of the selected nucleotide, then come in with a reporter oligonucleotide having a fluorescent label, then add ligase, and wherein the target oligonucleotide has at its 3′ end the polymorphic bases. For a typical OLA reaction with capture read out, the reagents can comprise: a target oligonucleotide containing two regions of complementarity; a first complementarity region of the target oligo is complementary to a region immediately adjacent to a single nucleotide polymorphism to be analyzed, a second complementarity region of the target oligonucleotide which is complementary to a capture oligonucleotide; a capture oligonucleotide that is covalently coupled to a mobile solid support; a reporter oligonucleotide complementary to the region overlapping the SNP and containing a means for readout, and a 3′ base on the strand opposite the SNP position; a ligase capable of ligating the reporter and the target if the base on the reporter that is opposite the SNP is complementary. In one embodiment of the method, the ligation reaction is then added to the capture-oligonucleotide-coupled mobile solid support and hybridization of the second complementarity region to the bead is allowed to occur under standard hybridization conditions. Readout of the reporter could be performed using a Luminex LX100-type machine.

The advantages to this system include the reduced number of bead sets needed to analyze many different SNPs, i.e., if given 100 bead colors, then one could synthesize only 100 capture oligonucleotides and use them over and over again in the different wells.

Thus, the present invention provides a method of detecting a result from an identification reaction (OLA) to identify a selected nucleotide in a target nucleic acid comprising:

a) hybridizing (i) a target oligonucleotide comprising a first complementarity region and a second complementarity region, wherein the first complementarity region comprises a region complementary to a section of the target nucleic acid that is directly 3′ of and adjacent to the selected nucleotide and the second complementarity region comprises a nucleic acid sequence complementary to a capture oligonucleotide, and (ii) a fluorescently labeled reporter oligonucleotide comprising a region complementary to a section of the target nucleic acid that is directly 5′ of and adjacent to the selected nucleotide, to a sample comprising the target nucleic acid, under hybridization conditions that allow specific hybridization between the first complementarity region of the target oligonucleotide and a region of the target nucleic acid complementary to the first complementarity region of the target oligonucleotide and that also allow specific hybridization between the reporter oligonucleotide and the section of the target nucleic acid complementary to the reporter oligonucleotide, to form a first hybridization product that defines a gap opposite the selected nucleotide;

b) adding an identified test nucleotide, a polymerase and a ligase, under conditions for polymerization and ligation to form a labeled product;

c) dissociating the hybridized nucleic acids;

d) isolating the labeled product by contacting the labeled product with a capture oligonucleotide that is covalently coupled to a mobile solid support, wherein the capture oligonucleotide comprises a nucleic acid sequence complementary to the second complementarity region of the target oligonucleotide, under hybridization conditions to form a second hybridization product; and

e) detecting the presence or absence of the label in the second hybridization product,

the presence of the label indicating polymerization of the identified test nucleotide to the target oligonucleotide and ligation of the labeled reporter oligonucleotide to the polymerized target oligonucleotide, and the identity of the identified test nucleotide indicating the identity of the nucleotide complementary to the selected nucleotide, thus identifying the selected nucleotide in the target nucleic acid.

As used throughout, the target nucleic acid can be genomic DNA treated to reduce viscosity, an oligonucleotide, a 16s ribosomal RNA, a 16S DNA, a PCR product, a DNA fragment, a restriction enzyme-generated DNA fragment, size-selected DNA, Bridge-amplified DNA, an RNA molecule, a cDNA molecule or a cRNA molecule.

The present invention further provides a method of determining a selected nucleotide polymorphism in genomic DNA treated to reduce viscosity comprising

a) performing an amplification of the genomic DNA using a first nucleic acid primer comprising a region complementary to a section of one strand of the nucleic acid that is 5′ of the selected nucleotide, and a second nucleic acid primer complimentary to a section of the opposite strand of the nucleic acid downstream of the selected nucleotide, under conditions for specific amplification of the region of the selected nucleotide between the two primers, to form a PCR product;

b) contacting the PCR product with a target oligonucleotide comprising a first complementarity region and a second complementarity region, wherein the first complementarity region is complementary to a section of one strand of the PCR product that is directly 5′ of and adjacent to the selected nucleotide, under hybridization conditions to form a first hybridization product;

c) performing a primer extension reaction with the first hybridization product and a detectably labeled, identified chain-terminating nucleotide under conditions for primer extension to form a primer extension product;

d) isolating the primer extension product by contacting the primer extension product with a capture oligonucleotide that is covalently coupled to a mobile solid support, wherein the capture oligonucleotide comprises a nucleic acid sequence complementary to the second complementarity region of the target oligonucleotide, under hybridization conditions to form an isolated second hybridization product;

e) detecting the presence or absence of a label incorporated into the second hybridization product, the presence of a label indicating the incorporation of the labeled chain-terminating nucleotide into the primer extension product, and the identity of the incorporated labeled chain-terminating nucleotide indicating the identity of the nucleotide complementary to the selected nucleotide; and

f) comparing the identity of the selected nucleotide with a non-polymorphic nucleotide,

a different identity of the selected nucleotide from that of the non-polymorphic nucleotide indicating a polymorphism of that selected nucleotide.

The present invention further provides a method of determining a selected nucleotide polymorphism in genomic DNA treated to reduce viscosity comprising

a) performing an amplification of the genomic DNA using as a primer an oligonucleotide comprising a first region having a T7 RNA polymerase promoter and a second region complementary to a section of one strand of the nucleic acid that is directly 5′ of the selected nucleotide, and using T7 RNA polymerase to amplify one strand into cRNA and using reverse transcriptase to amplify the second strand complementary to the cRNA strand, under conditions for specific amplification of the region of the nucleotide between the two primers, to form an amplification product;

b) contacting the amplification product with a target oligonucleotide comprising a first complementarity region and a second complementarity region, wherein the first complementarity region is complementary to a section of one strand of the PCR product that is directly 5′ of and adjacent to the selected nucleotide and wherein the second complementarity region is 5′ to the first complementarity region, under hybridization conditions to form a first hybridization product;

c) performing a primer extension reaction with the first hybridization product and a detectably labeled, identified chain-terminating nucleotide under conditions for primer extension to form a primer extension product;

d) isolating the primer extension product by contacting the primer extension product with a capture oligonucleotide that is covalently coupled to a mobile solid support, wherein the capture oligonucleotide comprises a nucleic acid sequence complementary to the second complementarity region of the target oligonucleotide, under hybridization conditions to form an isolated second hybridization product;

e) detecting the presence or absence of a label incorporated into the second hybridization product, the presence of a label indicating the incorporation of the labeled chain-terminating nucleotide into the primer extension product, and the identity of the incorporated labeled chain-terminating nucleotide indicating the identity of the nucleotide complementary to the selected nucleotide; and

f) comparing the identity of the selected nucleotide with a non-polymorphic nucleotide,

a different identity of the selected nucleotide from that of the non-polymorphic nucleotide indicating a polymorphism of that selected nucleotide.

The labeled chain-terminating nucleotide can be, for example, a 3′deoxynucleotide, a 3′deoxyribonucleotide, a thiol nucleotide derivative or a dideoxynucleotide. The amplification product can be in single-stranded form. Furthermore, one can design and synthesize some primers to sit just downstream of the target oligonucleotides.

The present method further provides a method of determining a selected nucleotide polymorphism in genomic DNA treated to reduce viscosity comprising

a) contacting the genomic DNA with a target oligonucleotide comprising a first complementarity region and a second complementarity region, wherein the first complementarity region is complementary to a section of one strand of the PCR product that is directly 5′ of and adjacent to the selected nucleotide and wherein the second complementarity region is 5′ to the first complementarity region, under hybridization conditions for forming a specific first hybridization product;

b) performing a primer extension reaction with the specific first hybridization product and a detectably labeled, identified chain-terminating nucleotide under conditions for primer extension;

c) isolating the primer extension product by contacting the primer extension product with a capture oligonucleotide that is covalently coupled to a mobile solid support, wherein the capture oligonucleotide comprises a nucleic acid sequence complementary to the second complementarity region of the target oligonucleotide, under hybridization conditions to form an isolated second hybridization product;

d) detecting the presence or absence of a label incorporated into the second hybridization product, the presence of a label indicating the incorporation of the labeled chain-terminating nucleotide into the hybridization product, and the identity of the incorporated labeled chain-terminating nucleotide indicating the identity of the nucleotide complementary to the selected nucleotide; and

e) comparing the identity of the selected nucleotide with a non-polymorphic nucleotide,

a different identity of the selected nucleotide from that of the non-polymorphic nucleotide indicating a polymorphism of that selected nucleotide.

The DNA can be in single-stranded form. The labeled chain-terminating nucleotide can be, for example, a 3′deoxynucleotide, a 3′deoxyribonucleotide, a thiol nucleotide derivative or a dideoxynucleotide. In such a method, the hybridization time should be of a length sufficient to allow hybridization of the first primer to the genomic DNA since the genomic DNA has not been amplified in this specific embodiment. Thus relatively long hybridization times may be utilized, such as, for example, 12 hours, 24 hours, 48 hours, as is known in the art for hybridization to genomic DNA (see, e.g., Sambrook, et al.).

In reactions utilizing a ligase, any selected ligase can be used, such as T4 DNA ligase. A thermostable ligase would be particularly useful. See, generally Wu and Wallace, Genomics 4: 560-569 (1989).

The invention further employs hybridization methods wherein two nucleic acids are hybridized to the sample nucleic acid but the step of ligation can be omitted and a match instead detected by fluorescence energy transfer between the two nucleic acids hybridized to the sample nucleic acid. The two hybridizing nucleic acids are designed such that the 3′ end of the target oligonucleotide is a test base, and when it is complementary to the polymorphic base, and a single wavelength of light is directed onto the sample, one can detect a transfer of energy, read as a second wavelength of light. A second reader can be employed for this detection of this second wavelength. Thus, the present invention provides a method of identifying a selected nucleotide in a target nucleic acid comprising

a) contacting the target nucleic acid with

i. a target oligonucleotide comprising a first complementarity region and a second complementarity region, wherein the first complementarity region is complementary to a section of the first nucleic acid that is directly 5′ of the selected nucleotide, wherein the target oligonucleotide terminates at its 3′ end in an identified test nucleotide positioned to base-pair with the selected nucleotide, and wherein the second complementarity region is 5′ to the first complementarity region, and

ii. a fluorescently labeled reporter oligonucleotide, wherein the reporter oligonucleotide comprises a region complementary to a section of the target nucleic acid that is directly 3′ of and adjacent to the selected nucleotide,

under hybridization conditions that allow the target nucleic acid and the target oligonucleotide to hybridize and the target nucleic acid and the reporter oligonucleotide to hybridize, thus forming a first hybridization product;

b) adding to the first hybridization product a ligase under ligation conditions;

c) isolating the first hybridization product by contacting the first hybridization product with a capture oligonucleotide that is covalently coupled to a mobile solid support, wherein the capture oligonucleotide comprises a nucleic acid sequence complementary to the second complementarity region of the target oligonucleotide, under hybridization conditions to form an isolated second hybridization product; and

d) detecting the presence or absence of the fluorescent label, after dissociation of the hybridized nucleic acids, in the second hybridization product, the presence of the label indicating the ligation of the labeled reporter oligonucleotide to the target oligonucleotide, and the identity of the test nucleotide in the target oligonucleotide indicating the identity of the nucleotide complementary to the selected nucleotide, thus identifying the selected nucleotide. The detection of the fluorescence energy transfer can be performed after dissociation of the hybridized nucleic acids.

The present invention additionally provides a method of identifying a selected nucleotide in a target nucleic acid comprising

a) contacting the target nucleic acid with

i. a target oligonucleotide linked at its 3′ end to a fluorescent label, wherein the target oligonucleotide comprises a first complementarity region that is complementary to a section of the target nucleic acid that is directly 3′ of the selected nucleotide, wherein the target oligonucleotide terminates at its 3′ end in a test nucleotide positioned to base-pair with the selected nucleotide, and wherein the target oligonucleotide has a second complementarity region 5′ of the first complementarity region, and

ii. a reporter oligonucleotide fluorescently labeled at its 5′ end, wherein the reporter oligonucleotide comprises a region complementary to a section of the target nucleic acid that is directly 5′ of and adjacent to the selected nucleotide,

under hybridization conditions that allow the target nucleic acid and the target oligonucleotide to hybridize and the target nucleic acid and the reporter oligonucleotide to hybridize, to form a first hybridization product;

b) isolating the first hybridization product by contacting the first hybridization product with a capture oligonucleotide that is covalently coupled to a mobile solid support, wherein the capture oligonucleotide comprises a nucleic acid sequence complementary to the second complementarity region of the target oligonucleotide, under hybridization conditions to form an isolated second hybridization product; and

c) detecting the presence or absence of fluorescent energy transfer between the fluorescent label at the 3′ end of the target oligonucleotide and the fluorescent label at the 5′ end of the reporter oligonucleotide in the second hybridization product,

the presence of fluorescent energy transfer indicating the hybridization of the identified test nucleotide to the target nucleic acid, and the identity of the hybridized test nucleotide in the target oligonucleotide indicating the identity of the nucleotide complementary to the selected nucleotide, thus identifying the selected nucleotide.

The present invention also provides a method for determining the sequence of a polymorphic base in a target nucleic acid which can utilize a kit comprising one or more of the following: a target oligonucleotide, wherein the target oligonucleotide comprises a first complementarity region and a second complementarity region 5′ of the first complementarity region, wherein the first complementarity region is complementary to a section of the target nucleic acid having a 3′ end adjacent to and directly 5′ of the polymorphic base on the target nucleic acid; a reporter oligonucleotide with an attached reporter moiety that is complementary to a region immediately adjacent to and 3′ of the polymorphic base of the target nucleic acid; the target oligonucleotide and the reporter oligonucleotide together defining a gap opposite the polymorphic base; a capture oligonucleotide that is covalently linked to a mobile solid support (such as a polystyrene-divinylbenzene bead), wherein the capture oligonucleotide comprises a nucleic acid sequence complementary to the second complementarity region of the target oligonucleotide; a nucleotide that is complementary to one of a set of two possible polymorphic bases; a polymerase, and a ligase, wherein the polymerase is able to polymerize the nucleotide across the gap if the nucleotide is complementary to the polymorphic base and wherein the ligase is able to ligate the newly polymerized nucleotide to the reporter oligonucleotide; and a means for detecting the reporter covalently linked to the bead. Further, the present invention provides a method of identifying a selected nucleotide in a target nucleic acid comprising

a) contacting the target nucleic acid with

i. a target oligonucleotide, wherein the target oligonucleotide comprises a first complementarity region and a second complementarity region 5′ of the first complementarity region, wherein the first complementarity region is complementary to a section of the target nucleic acid that is directly 3′ of and immediately adjacent to the selected nucleotide, and

ii. a reporter oligonucleotide fluorescently labeled, wherein the reporter oligonucleotide comprises a region complementary to a section of the second nucleic acid that is directly 5′ of and adjacent to the selected nucleotide,

under hybridization conditions that allow the target nucleic acid and the target oligonucleotide to form a hybridization product and the target nucleic acid and the reporter oligonucleotide to form a hybridization product, wherein the target nucleic acid, target oligonucleotide and reporter oligonucleotide form a hybridization product that defines a gap opposite the selected nucleotide;

b) adding an identified test nucleotide, a polymerase and a ligase, under conditions for polymerization and ligation;

c) isolating the first hybridization product by contacting the first hybridization product with a capture oligonucleotide that is covalently coupled to a mobile solid support, wherein the capture oligonucleotide comprises a nucleic acid sequence complementary to the second complementarity region of the target oligonucleotide, under hybridization conditions to form an isolated second hybridization product; and

d) detecting the presence or absence of the fluorescent label, after dissociation of the hybridized nucleic acids, in the second hybridization product,

the presence of the label indicating the polymerization of the test nucleic acid to the target oligonucleotide and ligation of the labeled reporter oligonucleotide to the target oligonucleotide linked to the mobile solid support, and the identity of the test nucleotide indicating the identity of the nucleotide complementary to the selected nucleotide, thus identifying the selected nucleotide.

The polymerase can preferably be a non-strand displacing polymerase. Further, it can be a thermostable polymerase. The ligase can be a DNA ligase. Further, it can be a thermostable ligase.

Furthermore, any of the herein described methods can be utilized in a method for quantitating expression of a selected nucleic acid in a sample. Thus, it can be used, for example, for differential gene expression wherein the expression of a selected gene is quantitated and compared to a standard or some other reference. For this method, a gene fragment from a region of interest or a region that distinguishes the gene (or allele or haplotype or polymorphism) of interest is selected for use as the first complementarity region of a target oligonucleotide; message (e.g., RNA, cDNA, cRNA) is hybridized to the target oligonucleotide, and fluorescence is quantitated by performing a primer extension reaction, a ligase reaction or a hybridization/fluorescence energy transfer reaction, such as that described herein. A corresponding capture oligonucleotide (complementary to a second complementarity region utilized in the target oligonucleotide) linked to a mobile solid support is utilized to capture the reaction product. The first complementarity region of a target oligonucleotide can comprise a region complementary to a section of the selected nucleic acid unique to the nucleic acid. A standard, such as that from a normal subject, or a diseased/afflicted subject, or a particular tissue or organ, or a particular species, can be used as a comparison reference to draw conclusions regarding the quantity detected in the sample.

Thus the present invention provides a method of quantitating expression of a selected nucleic acid in a sample comprising

a) contacting (i) message nucleic acid isolated from a selected source with (ii) a target oligonucleotide, wherein the target oligonucleotide comprises a first complementarity region and a second complementarity region 5′ of the first complementarity region, wherein the first complementarity region comprises a region complementary to a section of the selected nucleic acid;

b) performing a selected identification reaction with the first hybridization product to determine the identity of the selected nucleotide wherein a selectively labeled detection product comprising the second complementarity region of the target oligonucleotide can be formed;

c) isolating the detection product by contacting the detection product with a capture oligonucleotide that is covalently coupled to a mobile solid support, wherein the capture oligonucleotide comprises a nucleic acid sequence complementary to the second complementarity region of the target oligonucleotide, under hybridization conditions to form an isolated hybridization product; and

d) quantitating the fluorescence in the isolated hybridization product,

the quantity of fluorescence indicating the quantity of the selected nucleic acid in the sample.

For any of these methods described herein, a sample can be, for example, any body sample that contains message, such as organ tissue and/or cells, such as blood, red or white blood cells, bone marrow, liver, kidney, brain, skin, heart, lung, spleen, pancreas, gall bladder, muscle, neural cells, neurons, precursor cells, ovaries, testicles, uterus, glands.

Additionally provided are kits for detecting a single base polymorphism, wherein a kit comprises a detectably tagged mobile solid support, such as a polystyrene-divinylbenzene bead, and one to four modified (chain-terminating) nucleotide(s), such as a 3′ deoxynucleotide,m, a 3′ deoxyribonucleotide, a thiol derivative, or a dideoxynucleotide. The kit can additionally comprise a polymerase, and in particular, a polymerase that preferentially incorporates the modified nucleotide. The kit can additionally comprise a ligase. The kit can also comprise one or more fluorescent label for labeling the nucleic acid(s). For genomic DNA uses, the kit can further comprises a DNase for reducing the viscosity of the DNA. The kit can further contain an array of combinations of dinucleotides and/or a collection of combinations of trinucleotides. Instead of chain-terminating nucleotides, the kits can comprise other reporter probes and labels for use in oligonucleotide ligation assays, allele-specific polymerase assays, cleavase signal release reactions, or polymerase repair reactions.

In one embodiment, the invention provides a method of detecting a result from an identification reaction to identify a selected nucleotide in a target nucleic acid comprising:

a. contacting a target oligonucleotide comprising a first complementarity region and a second complementarity region, wherein the second complementarity region is 5′ of the first complementarity region and wherein the first complementarity region comprises a region complementary to a section of the target nucleic acid that is directly 3′ of and adjacent to the selected nucleotide, with a sample comprising the target nucleic acid, under hybridization conditions that allow the formation of a first hybridization product;

b. performing, in the presence of a selectively labeled reporter probe, a selected identification reaction with the first hybridization product to determine the identity of the selected nucleotide, wherein a selectively labeled detection product comprising the target oligonucleotide and the reporter probe can be formed;

c. isolating the detection product by contacting the detection product with a capture oligonucleotide that is covalently coupled directly or indirectly to a mobile solid support, wherein the capture oligonucleotide comprises a nucleic acid sequence complementary to the second complementarity region of the target oligonucleotide, under hybridization conditions to form a second hybridization product; and

d. detecting the label of the labeled detection product in the second hybridization product,

the presence of the label indicating the identity of the selected nucleotide in the target nucleic acid.

As used throughout, the capture oligonucleotide (also referred to herein as the cZipCode) can have a GC content of about 50% or greater. Also, the capture oligonucleotide can have a Tₘ of about 60 to 70° C. Preferably, the capture oligonucleotide comprises a sequence not present in a cell that contains the target nucleic acid of interest. For example, the target nucleic acid can be a sequence present in mammalian cells and the capture oligonucleotide can comprise an oligonucleotide sequence present in a bacterium. More specifically, the capture oligonucleotide can comprise an oligonucleotide sequence present in Mycobacterium tuberculosis.

Also, as used throughout, the capture oligonucleotide can further comprise a 5′ amine group. The capture oligonucleotide can further comprise a luciferase cDNA. For example, the luciferase cDNA can have the sequence of CAGGCCAAGTAACTTCTTCG (SEQ ID NO:59).

Also, as used in the various embodiments of the invention, the capture oligonucleotide can be directly or indirectly coupled to the mobile solid support. More specifically, the capture oligonucleotide can be indirectly coupled to the mobile solid support by a carbon spacer. The capture oligonucleotide can coupled at either its 5′ or 3′ end to the mobile solid support. Accordingly, the label attached to the oligonucleotide that hybridizes to the probe can be directed toward or away from the mobile solid support without hinderance to the detection of the label.

The second complementarity region of the target oligonucleotide, as used in the various embodiments of the present invention, preferably comprises a nucleic acid of at least 8, 10, 15, or 25 nucleotides. More specifically, the second complementarity region of the target oligonucleotide can comprises a nucleic acid having the sequence selected from the group consisting of SEQ ID NO:1-58 as show in Table 1.

The identification reaction of the present invention can be a single base chain extension reaction. Specifically, the single base chain extension reaction comprises performing a primer extension reaction with the first hybridization product; wherein the detectably labeled reporter probe comprises an identified, chain-terminating nucleotide under conditions for primer extension; and wherein the presence of a label in the second hybridization product indicates the incorporation of the labeled nucleotide into the first hybridization product, the identity of the incorporated labeled nucleotide indicating the identity of the nucleotide complementary to the selected nucleotide, thus identifying the selected nucleotide in the target nucleic acid. The chain-terminating nucleotide can be a 3′deoxynucleotide, a 3′deoxyribonucleotide, a thiol nucleotide derivative or a dideoxynucleotide. When the chain terminating nucleotide is a dideoxynucleotide, the primer extension can be performed in the presence of either (1) one labeled, identified dideoxynucleotide and three different, non-labeled dideoxynucleotides; (2) one labeled, identified dideoxynucleotide and two different, non-labeled dideoxynucleotides; (3) one labeled, identified dideoxynucleotide and one different, non-labeled dideoxynucleotides; or (4) one labeled, identified dideoxynucleotide and in the absence of any different, non-labeled dideoxynucleotides. The label of the chain-terminating nucleotide can be selected from the group consisting of a hapten, radiolabel, and fluorescent label.

As used throughout, “label” refers to haptens that provide a means for labeling, radiolabels, and fluorescent labels.

In alternative embodiments of the present invention, the identification reaction can be an oligonucleotide ligation reaction. Specifically, the oligonucleotide ligation reaction comprises performing a ligation reaction between the target oligonucleotide and the reporter probe; wherein the selectively labeled reporter probe comprises a sequence that is complementary to a section of the target nucleic acid directly 5′ the selected nucleotide and that terminates at its 3′ end in an identified test nucleotide positioned to base-pair with the selected nucleotide of the target nucleic acid, under conditions for ligation; and wherein the detection comprises detecting the presence or absence of a label incorporated into the second hybridization product, the presence of a label indicating the incorporation of the labeled reporter probe in the reaction product, and the identity of the incorporated labeled reporter probe indicating the identity of the nucleotide complementary to the selected nucleotide, thus identifying the selected nucleotide in the target nucleic acid. In the oligonucleotide ligation reaction, the reporter probe can comprise one or more nucleotides and have a 5′ phosphate group. Furthermore, the reporter probe further comprises a 3′ label. Preferably, the reporter probe is an oligonucleotide. More preferably, the oligonucleotide of the reporter probe is an 8-mer. An advantage of OLA is the ability to read alleles from a given SNP in one tube (with SBCE, each base querried requires analysis in a separate tube when using ddNTP terminators labeled with one fluorochrome). Additionally, in an OLA reaction, there is no requirement to remove dNTPs from the PCR preparation. In contrast, an advantage of SBCE is that separate reporter probes do not need to be designed for each single nucleotide polymorphism.

In other embodiments, the identification reaction can be an allelle-specific polymerization reaction (i.e., minisequencing). The allelle-specific polymerization reaction can comprise performing a polymerization reaction with a non-proof reading polymerase, wherein a primer for the reaction comprises the first complementarity region of the target oligonucleotide, wherein the reporter probe comprises one or more selectively labeled deoxynucleotides, and wherein the detection comprises detecting the presence or absence of a label incorporated into the second hybridization product, the presence of the label indicating the extension of the primer and the identity of the label indicating the nucleotide complementary to the selected nucleotide, thus identifying the selected nucleotide in the target nucleic acid. The nucleic acid primer can be an oligonucleotide, a PCR product, a DNA fragment, an RNA molecule, a cDNA molecule, a cRNA molecule, or genomic DNA.

As used throughout, the mobile solid support is preferably a bead, and more preferably a polystyrene-divinylbenzene bead. In one embodiment, the bead can be strepavidin-coated, and the capture oligonucleotide can be biotinylated, and thereby the biotin on the capture probe and the strepavidin on the bead providing a high affinity binding between the capture probe and the bead. One skilled in the art would recognize that, when biotin is used as a means of labeling the reporter probe and as a means of binding the capture probe to the mobile solid support, the strepavidin on the mobile solid support must be saturated with biotin to prevent direct binding of the biotin of the reporter probe to the mobile solid support.

As discussed above and as used in the various embodiments of the invention, the mobile solid support can be detectably tagged with a dye, radiolabel, magnetic tag, or a Quantum Dot® (Quantum Dot Corp.). The detectable tag can be detected either by passing the mobile solid support over a laser detection device capable of detecting the detectable tag or by placing the mobile solid support on a two-dimensional surface and passing a laser detection device capable of detecting/distinguishing the detectable tag over the solid support. Preferably, the same laser detection device detects or distinguishes the label of the labeled detection product and the detectable tag of the mobile solid support in the same second hybridization product. When radiolabels or radiotags are used in the present method, an alternative detection device is used. For example, radiotags or radiolabels can be detected by embedding the sample to be read in scintillation fluid and using a non-laser detector.

As numerous labels and detectable tags can be detected by the same detection device in the same sample so long as the detection device can differentiate the signal of each label or detectable tag, opportunities for multiplexing are available. In the various embodiments of the present invention, the mobile solid support for example, can include a set of beads having different detectable tags and selected capture probes selected for that detectable tag. Analytical readout platforms include also include both solid supports (gels, chips, glass slides) and mobile supports such as mass-spectrometry and electrophoresis (gel and capillary).

To further multiplex within the same sample, the methods of the present invention, as provided throughout, can further comprise performing the selected identification reaction in the presence of more than one reporter probe, wherein each reporter probe comprises a different detectable label and a different nucleotide complementary to the selected nucleotide of the target nucleic acid, to produce detection products with different labels, and detecting the different labels of the labeled detection products in the second hybridization products, the presence of each label indicating the identity of each selected nucleotide in the target nucleic acid. Optionally, the different labels of the labeled detection products in the second hybridization products can be quantified, the quantity of the different labels indicating the relative occurrence of each selected nucleotide in the target nucleic acid.

As another embodiment of multiplexing, more than one capture oligonucleotide can be covalently coupled to the mobile solid support and each second hybridization product can comprise one or more labels. Thus, for example, in a set of beads having different detectable tags, one bead could have two or more selected capture probes. Each bead could have its own detectable tag and, upon hybridization of the capture probes with the detection products, have two specific labels associated with the same bead. For example, a bead having a specific fluorescence could have both a fluorescein and a rhodamine label attached. The detection device could differentiate all three signals on one bead and could read similar signals are beads having different fluorescent wavelengths. The multiplexing method could further comprise quantifying the different labels of the labeled detection products in the second hybridization products, the quantity of the different labels indicating the relative occurrence of each selected nucleotide in the target nucleic acid.

As an alternative embodiment, the present invention provides a method of detecting a result from an identification reaction to identify a selected nucleotide in a target nucleic acid can alternatively comprise:

a. contacting a target oligonucleotide comprising a first complementarity region and a second complementarity region, wherein the second complementarity region is 3′ of the first complementarity region and wherein the first complementarity region comprises a region complementary to a section of the target nucleic acid that is directly 5′ of and adjacent to the selected nucleotide, with a sample comprising the target nucleic acid, under hybridization conditions that allow the formation of a first hybridization product;

b. performing, in the presence of a selectively labeled reporter probe, a selected identification reaction with the first hybridization product to determine the identity of the selected nucleotide, wherein a selectively labeled detection product comprising the target oligonucleotide and the reporter probe can be formed;

c. isolating the detection product by contacting the detection product with a capture oligonucleotide that is covalently coupled directly or indirectly to a mobile solid support, wherein the capture oligonucleotide comprises a nucleic acid sequence complementary to the second complementarity region of the target oligonucleotide, under hybridization conditions to form a second hybridization product; and

d. detecting the label of the labeled detection product in the second hybridization product,

the presence of the label indicating the identity of the selected nucleotide in the target nucleic acid. In this embodiment, the capture oligonucleotide can be coupled at either its 5′ or 3′ end to the mobile solid support. Preferably, the identification reaction using this 3′ to 5′ directionality is an oligonucleotide ligation reaction. For example, the oligonucleotide ligation reaction can comprise performing a ligation reaction between the target oligonucleotide and the reporter probe; wherein the selectively labeled reporter probe comprises a sequence that is complementary to a section of the target nucleic acid directly 3′ the selected nucleotide and that terminates at its 5′ end in an identified test nucleotide positioned to base-pair with the selected nucleotide of the target nucleic acid, under conditions for ligation; and wherein the detection comprises detecting the presence or absence of a label incorporated into the second hybridization product, the presence of a label indicating the incorporation of the labeled reporter probe in the reaction product, and the identity of the incorporated labeled reporter probe indicating the identity of the nucleotide complementary to the selected nucleotide, thus identifying the selected nucleotide in the target nucleic acid. In this embodiment, the reporter probe comprises one or more nucleotides and has a 3′ phosphate group. The reporter probe can further comprises a 5′ label.

As a means of multiplexing, the present invention further provides a method of detecting a result from an identification reaction to identify one or more selected nucleotides in one or more target nucleic acids comprising:

a. contacting one or more specific target oligonucleotides, wherein each target oligonucleotide comprises a first specific complementarity region and a second specific complementarity region, wherein the second complementarity region of each target oligonucleotide is 5′ of the first complementarity region and wherein the first complementarity region of each target oligonucleotide comprises a sequence that is complementary to a section of the target nucleic acid directly 3′ of the selected nucleotide and that terminates at its 3′ end in an identified test nucleotide positioned to base-pair with the selected nucleotide of the target nucleic acid, with a sample comprising one or more target nucleic acids, under hybridization conditions, to form first hybridization products;

b. performing, in the presence of one or more selectively labeled reporter probes, a selected identification reaction with the first hybridization products, wherein selectively labeled detection products comprising the first complementarity region of the target oligonucleotides and the reporter probes can be formed;

c. isolating the detection products by contacting the detection products, under hybridization conditions to form second hybridization products, with specific capture oligonucleotides that are covalently coupled directly or indirectly to specific detectably tagged mobile solid supports, wherein each capture oligonucleotide comprises a nucleic acid sequence complementary to a second complementarity region of a specific target oligonucleotide and wherein the detectable tag is specific for each capture oligonucleotide; and

d. detecting the labels of the labeled detection product in the second hybridization product and the detectable tags of the mobile solid support in the same second hybridization product,

the presence of the label and the specific detectable tag in the same second hybridization product indicating the identity of the selected nucleotides in the target nucleic acid. The identification reaction can be a single base chain extension reaction. Specifically, the single base chain extension reaction comprises performing a primer extension reaction with the first hybridization products; wherein each detectably labeled reporter probe comprises an identified, chain-terminating nucleotide under conditions for primer extension; and wherein the presence of a selected label in the second hybridization product indicates the incorporation of the labeled nucleotide into the first hybridization product, the identity of the incorporated labeled nucleotide indicating the identity of the nucleotide complementary to the selected nucleotide, thus identifying the selected nucleotide in the target nucleic acid. Alternatively, the identification reaction can be an oligonucleotide ligation reaction. Specifically, the oligonucleotide ligation reaction comprises performing a ligation reaction between the target oligonucleotides and the reporter probes. Alternatively, the identification reaction is an allelle-specific polymerization reaction, wherein the allelle-specific polymerization reaction comprises performing a polymerization reaction with a non-proof reading polymerase, wherein each primer for the reaction comprises the first complementarity region of the target oligonucleotide, wherein the reporter probe comprises one or more selectively labeled deoxynucleotides, and wherein the detection comprises detecting the presence or absence of a label incorporated into the second hybridization product, the presence of the label indicating the extension of the primer and the identity of the label indicating the nucleotide complementary to the selected nucleotide, thus identifying the selected nucleotide in the target nucleic acid.

In the present invention, the detection device detects or distinguishes the various labels of the labeled detection products and the various detectable tags of the mobile solid support in the same second hybridization products. Optionally, the labels and specific detectable tags in the second hybridization products can be quantified, the quantity of the labels and specific detectable tags in the second hybridization products indicating the relative occurrence of each selected nucleotide in the target nucleic acid.

The same embodiment can be practiced using a reversed 3′ to 5′ directionality. Thus, the present invention provides a method of detecting a result from an identification reaction to identify one or more selected nucleotides in one or more target nucleic acids comprising:

a. contacting one or more specific target oligonucleotides, wherein each target oligonucleotide comprises a first specific complementarity region and a second specific complementarity region, wherein the second complementarity region of each target oligonucleotide is 3′ of the first complementarity region and wherein the first complementarity region of each target oligonucleotide comprises a sequence that is complementary to a section of the target nucleic acid directly 5′ of the selected nucleotide and that terminates at its 5′ end in an identified test nucleotide positioned to base-pair with the selected nucleotide of the target nucleic acid, with a sample comprising one or more target nucleic acids, under hybridization conditions, to form first hybridization products;

b. performing, in the presence of one or more selectively labeled reporter probes, a selected identification reaction with the first hybridization products, wherein selectively labeled detection products comprising the first complementarity region of the target oligonucleotides and the reporter probes can be formed;

c. isolating the detection products by contacting the detection products, under hybridization conditions to form second hybridization products, with specific capture oligonucleotides that are covalently coupled directly or indirectly to specific detectably tagged mobile solid supports, wherein each capture oligonucleotide comprises a nucleic acid sequence complementary to a second complementarity region of a specific target oligonucleotide and wherein the detectable tag is specific for each capture oligonucleotide; and

d. detecting the labels of the labeled detection product in the second hybridization product and the detectable tags of the mobile solid support in the same second hybridization product,

the presence of the label and the specific detectable tag in the same second hybridization product indicating the identity of the selected nucleotides in the target nucleic acid. Each capture oligonucleotide can be coupled at either its 5′ or 3′ end to the mobile solid support. Preferably, the identification reaction is an oligonucleotide ligation reaction. More preferably, the reporter probe comprises one or more nucleotides and has a 5′ phosphate group and further comprises a 5′ label. The 5′ phosphate group is required as a substrate for specific ligase enzymes.

The present invention also provides a method of determining one or more selected nucleotide polymorphisms in genomic DNA comprising

a′. performing an amplification of the genomic DNA using a first nucleic acid primer comprising a region complementary to a section of one strand of the nucleic acid that is 5′ of the selected nucleotide, and a second nucleic acid primer complimentary to a section of the opposite strand of the nucleic acid downstream of the selected nucleotide, under conditions for specific amplification of the region of the selected nucleotide between the two primers, to form a PCR product;

a″ performing an amplification of the genomic DNA using as a primer an oligonucleotide comprising a first region having a T7 RNA polymerase promoter and a second region complementary to a section of one strand of the nucleic acid that is directly 5′ of the selected nucleotide, and using T7 RNA polymerase to amplify one strand into cRNA and using reverse transcriptase to amplify the second strand complementary to the cRNA strand, under conditions for specific amplification of the region of the nucleotide between the two primers, to form a cRNA amplification product; or

a′″. treating genomic DNA to decrease viscosity; and

b. contacting a sample comprising one or more PCR products, one or more cRNA amplification products, or treated genomic DNA with one or more specific target oligonucleotides, wherein each target oligonucleotide comprises a first specific complementarity region and a second specific complementarity region, wherein the second complementarity region of each target oligonucleotide is 5′ of the first complementarity region, and wherein the first complementarity region of each target oligonucleotide comprises a sequence that is complementary to a section of the target nucleic acid directly 5′ of the selected nucleotide and that terminates at its 3′ end in an identified test nucleotide positioned to base-pair with a selected nucleotide of the PCR products, cRNA amplification products, or treated genomic DNA, under hybridization conditions, to form first hybridization products;

c. performing, in the presence of one or more selectively labeled reporter probes, a selected identification reaction with the first hybridization products, wherein selectively labeled detection products comprising the first complementarity region of the target oligonucleotides and the reporter probes can be formed;

d. isolating the detection products by contacting the detection products, under hybridization conditions to form a second hybridization product, with specific oligonucleotides that are covalently coupled directly or indirectly to specific detectably tagged mobile solid supports, wherein each capture oligonucleotide comprises a nucleic acid sequence complementary to a second complementarity region of a specific target oligonucleotide and wherein the detectable tag is specific for each capture oligonucleotide; and

e. detecting the label of the labeled detection product in the second hybridization product and the detectable tag of the mobile solid support in the same second hybridization product, the presence of the label and the specific detectable tag in the same second hybridization product indicating the identity of the selected nucleotide in the specific PCR products, cRNA amplification products, or treated genomic DNA; and

f. comparing the identities of the identified nucleotides with a non-polymorphic nucleotide,

a different identity of the identified nucleotide from that of the non-polymorphic nucleotide indicating one or more polymorphisms in the genomic DNA. The identification reaction can be a single base chain extension reaction, is an oligonucleotide ligation reaction, or an allelle-specific polymerization reaction, as described above. Also as described above, numerous opportunities for multiplexing can be exploited, including, for example, the method in which more than one capture oligonucleotide covalently coupled to the mobile solid support and wherein each second hybridization product can comprise one or more labels. Accordingly, the method can further comprise quantifying the labels and specific detectable tags in the second hybridization products, the quantity of the labels and specific detectable tags in the same second hybridization products indicating the relative occurrence of each selected nucleotide in the target nucleic acid.

The present invention further provides a method of detecting results from a cleavase/signal release reaction to identify one or more selected nucleotides in a target nucleic acid comprising:

a. contacting a sample comprising the target nucleic acid with (i) one or more signal probes, wherein each signal probe comprises a first complementarity region and a selected second complementarity region that is specific for a test nucleotide, wherein the second complementarity region is 5′ of the first complementarity region and comprises a donor fluorophore, and wherein the first complementarity region comprises (a) a sequence that is complementary to a section of the target nucleic acid that is directly 5′ of the selected nucleotide, (b) the test nucleotide at its 5′ end that is positioned to base-pair with the selected nucleotide of the target nucleic acid, and (c) a quenching fluorophore that is located 3′ to the identified test nucleotide and (ii) more than one invader oligonucleotide, wherein each invader oligonucleotide comprises (a) a sequence that is complementary to a section of the target nucleic acid that is directly 3′ of the selected nucleotide and (b) the identified test nucleotide at its 5′ end that is positioned to base-pair with the selected nucleotide of the target nucleic acid, under hybridization conditions that allow the formation of overlapping hybridization products between the first complementarity region of the signal probes and the section of the target nucleic acid complementary to the first complementarity region of the signal probes and between the invader oligonucleotides and the complementary section of the target nucleic acid, to form the overlapping hybridization products, wherein the overlapping hybridization products overlap at the selected nucleotide;

b. performing specific cleavage reactions comprising contacting the overlapping hybridization products with a nuclease that specifically cleaves the overlapping hybridization products formed when the identified test nucleotide and selected nucleotide are complementary, and releasing detection products comprising the specific second complementary regions and the identified test nucleotide of the first complementarity region of the signal probes;

c. isolating the detection products by contacting the detection products, under hybridization conditions to form non-overlapping second hybridization products, with specific capture oligonucleotides that are covalently coupled directly or indirectly to specific detectably tagged mobile solid supports, wherein each capture oligonucleotide comprises a nucleic acid sequence complementary to a specific second complementarity region of a specific signal probe and wherein the detectable tag is specific for each capture oligonucleotide; and

d. detecting the presence of the donor fluorophore and the absence of the quenching fluorophore and the presence of the detectable tags of the mobile solid support in the same in the non-overlapping hybridization products,

the presence of the specific detectable tag and the donor fluorophore and the absence of the quenching fluorophore indicating the identity of the selected nucleotide in the target nucleic acid. Optionally, the method can further comprise repetitions of steps (a) and (b) above to increase the level of detection product or products. As described above, each capture oligonucleotide can be coupled at either its 5′ or 3′ end to the mobile solid support. The method can further comprise quantifying the occurrence of specific detectable tags and donor fluorophores and the absence of quenching fluorophores in the same non-overlapping hybridization products indicating the relative occurrence of each selected nucleotide in the target nucleic acid.

The method of detecting results from a cleavase/signal release reaction to identify one or more selected nucleotides in a target nucleic acid comprising the reaction in the opposite 3′ to 5′ directionality as follows:

a. contacting a sample comprising the target nucleic acid with (i) one or more signal probes, wherein each signal probe comprises a first complementarity region and a selected second complementarity region that is specific for a test nucleotide, wherein the second complementarity region is 3′ of the first complementarity region and comprises a donor fluorophore, and wherein the first complementarity region comprises (a) a sequence that is complementary to a section of the target nucleic acid that is directly 3′ of the selected nucleotide, (b) the test nucleotide at its 3′ end that is positioned to base-pair with the selected nucleotide of the target nucleic acid, and (c) a quenching fluorophore that is located 5′ to the identified test nucleotide and (ii) more than one invader oligonucleotide, wherein each invader oligonucleotide comprises (a) a sequence that is complementary to a section of the target nucleic acid that is directly 5′ of the selected nucleotide and (b) the identified test nucleotide at its 3′ end that is positioned to base-pair with the selected nucleotide of the target nucleic acid, under hybridization conditions that allow the formation of overlapping hybridization products between the first complementarity region of the signal probes and the section of the target nucleic acid complementary to the first complementarity region of the signal probes and between the invader oligonucleotides and the complementary section of the target nucleic acid, to form the overlapping hybridization products, wherein the overlapping hybridization products overlap at the selected nucleotide;

b. performing specific cleavage reactions comprising contacting the overlapping hybridization products with a nuclease that specifically cleaves the overlapping hybridization products formed when the identified test nucleotide and selected nucleotide are complementary, and releasing detection products comprising the specific second complementary regions and the identified test nucleotide of the first complementarity region of the signal probes;

c. isolating the detection products by contacting the detection products, under hybridization conditions to form non-overlapping second hybridization products, with specific capture oligonucleotides that are covalently coupled directly or indirectly to specific detectably tagged mobile solid supports, wherein each capture oligonucleotide comprises a nucleic acid sequence complementary to a specific second complementarity region of a specific signal probe and wherein the detectable tag is specific for each capture oligonucleotide; and

d. detecting the presence of the donor fluorophore and the absence of the quenching fluorophore and the presence of the detectable tags of the mobile solid support in the same in the non-overlapping hybridization products,

the presence of the specific detectable tag and the donor fluorophore and the absence of the quenching fluorophore indicating the identity of the selected nucleotide in the target nucleic acid.

The present invention provides a method of detecting results from a polymerase/repair reaction to identify selected nucleotides in a target nucleic acid comprising:

a. contacting a sample comprising the target nucleic acid with (i) one or more signal probes, wherein each signal probe comprises a first complementarity region and a selected second complementarity region that is specific for a test nucleotide, wherein the second complementarity region is 3′ of the first complementarity region, and wherein the first complementarity region comprises (a) a sequence that is complementary to a section of the target nucleic acid that is directly 5′ of the selected nucleotide, (b) the identified test nucleotide at the 5′ end of the signal probe, wherein the test nucleotide is positioned to base-pair with the selected nucleotide of the target nucleic acid, (c) a thiol site located 3′ of the test nucleotide, (d) a donor fluorophore that is located 3′ to the thiol site, (d) a quenching fluorophore that is located 5′ to the thiol site and 3′ to the test nucleotide, under hybridization conditions that allow the formation of first hybridization products between the first complementarity region of the signal probes and the section of the target nucleic acid complementary to the first complementarity region of the signal probes;

b. performing a polymerase/repair reaction comprising contacting the first hybridization products with a Taq polymerase that cleaves the signal probes at the thiol site when the test nucleotide and the selected nucleotide are complementary and releases detection products comprising the second complementary region and the portion of the first complementary region of the signal probes that contain the donor fluorophore but lack the quenching fluorophore;

c. isolating the detection products by contacting the detection products, under hybridization conditions to form second hybridization products, with specific capture oligonucleotides that are covalently coupled directly or indirectly to specific detectably tagged mobile solid supports, wherein each capture oligonucleotide comprises a nucleic acid sequence complementary to a specific second complementarity region of a specific signal probe and wherein the detectable tag is specific for each capture oligonucleotide; and

d. detecting the presence of the donor fluorophore, the absence of the quenching fluorophore, and the presence of the specific detectable tags of the mobile solid support in the same second hybridization products,

the presence of the specific detectable tag and the donor fluorophore and the absence of the quenching fluorophore indicating the identity of the selected nucleotides in the target nucleic acid. The method can further comprise repetitions of steps (a) and (b) above to increase the amount of detection product. The method can also further comprise quantifying the occurrence of specific detectable tags and donor fluorophores and the absence of quenching fluorophores in the same non-overlapping hybridization products indicating the relative occurrence of each selected nucleotide in the target nucleic acid.

The present invention also provides a method of detecting one or more selected microbial contaminants in a sample comprising:

a. contacting the sample with one or more target oligonucleotides, wherein each target oligonucleotide comprises a first complementarity region and a second complementarity region, wherein the first complementarity region comprises a region complementary to a section of a nucleic acid that is specific to a selected microbial contaminant and wherein the second complementarity region comprises a region complementary to a specific labeled reporter probe, under hybridization conditions that allow the formation of hybridization products between the first complementarity region of the target oligonucleotides and a region of the microbial nucleic acid complementary to the first complementarity region of the target oligonucleotide, to form first hybridization products;

b. performing, in the presence of one or more labeled reporter probes, a selected identification reaction with the first hybridization products, wherein selectively labeled detection products can be formed and wherein each detection product comprises the second complementary of a specific target oligonucleotide and a label;

c. isolating the detection products by contacting the detection products with specific capture oligonucleotides that are covalently coupled directly or indirectly to specific detectably tagged mobile solid supports, wherein each capture oligonucleotide comprises a nucleic acid sequence complementary to a second complementarity region of a specific target oligonucleotide and wherein the detectable tag is specific for each capture oligonucleotide; and

d. detecting the labels of the labeled detection product in the second hybridization product and the detectable tags of the mobile solid support in the same second hybridization product,

the presence of the label and the specific detectable tag in the same second hybridization product indicating the identity of microbial contaminants in the sample. The selected microbial contaminants can include, but are not limited to, S. aureus, B. cepacia, E. coli, and Pseudomonas. The contaminants can be identified in the same sample using the multiplexing techniques described above. It is understood that the identification reaction can be oligonucleotide ligation reaction, single base chain extension, allelle-specific polymerization reaction, a cleavase/signal release reaction, or a polymerase/repair reaction as described in detail above. It is further understood that the microbial DNA can be amplified, for example, by PCR, prior to the identification reaction. The samples that can be tested for microbial contaminants include but are not limited to food samples, drug/pharmacological samples, blood samples, urine samples, and various reagents for use in food and drug preparation.

To detect S. aureus contaminant in a sample, the method can be practiced using the first complementarity region complementary to a section of a nucleic acid that is specific to S. aureus having the nucleic acid sequence GCCGGTGGAGTAACCTTTTAG (SEQ ID NO:60) or GCCGGTGGAGTAACCTTTTAGG (SEQ ID NO:61).

To detect B. cepacia in a sample, the method can be practiced using the first complementarity region complementary to a section of a nucleic acid that is specific to B. cepacia having the nucleic acid sequence CTGAGAGGCGGGAGTGCT (SEQ ID NO:62) or CTGAGAGGCGGGAGTGCTC (SEQ ID NO:63).

To detect a microbial contaminant that is either E. coli or Pseudomonas, the method can be practiced, wherein the first complementarity region complementary to a section of a nucleic acid that is specific to E. coli or Pseudomonas has the nucleic acid sequence AATACCGCATA (SEQ ID NO:64) or AATACCGCATA C/A (SEQ ID NO:65).

To detect a microbial contaminant that is either Pseudomonas or B. cepacia, the method can be practiced, wherein the first complementarity region complementary to a section of a nucleic acid that is specific to Pseudomonas or B. cepacia has the nucleic acid sequence of AATACCGCATACG (SEQ ID NO:66) or AATACCGCATACG T/A (SEQ ID NO:67).

The following documents provide information regarding various technologies: PCT publication WO 9714028 (Luminex Corp.).

Australian patent AU 9723205 (based on WO 9735033 (Sep. 25, 1997)) (Molecular Tool Inc.)

European patent publication EP 754240 (based on WO 9521271) (Molecular Tool Inc.)

European patent publication EP 736107 (based on WO 9517524) (Molecular Tool Inc.)

U.S. Pat. No. 5,610,287 (Mar. 11, 1997) (Molecular Tool Inc.)

European patent publication EP 726905 (based on WO 9512607) (Molecular Tool Inc.)

U.S. Pat. No. 5,518,900 (Jul. 21, 1994) (Molecular Tool Inc.)

European patent publication EP 576558 (based on WO 9215712) (Molecular Tool Inc.)

### EXAMPLE 1

Multiplexed Single Nucleotide Polymorphism Genotyping by Oligonucleotide Ligation and Flow Cytometry

In this high throughput method for single nucleotide polymorphism (SNP) genotyping, an oligonucleotide ligation assay (OLA) and flow cytometric analysis of fluorescent microspheres was used by adding a fluoresceinated oligonucleotide reporter probe (or reporter sequence) a target oligonucleotide by OLA. The target oligonucleotides were designed to hybridize both to genomic ‘targets’ amplified by polymerase chain reaction and to a separate complementary DNA sequence that has been coupled to a microsphere. These sequences on the target oligonucleotides that hybridize to a sequence coupled to the microsphere are called ‘ZipCodes’. The OLA-modified target oligonucleotides are hybridized to ZipCode complement-coupled microspheres. The use of microspheres with different ratios of red and orange fluorescence makes a multiplexed format possible where many SNPs may be analyzed in a single tube. Flow cytometric analysis of the microspheres simultaneously identifies both the microsphere type and the fluorescent green signal associated with the SNP genotypying. Multiplexed genotyping of seven CEPH DNA samples for nine SNP markers located near the ApoElocus on chromosome 19 was performed, and the results were verified with genotyping by sequencing in all cases. A set of fluorescent latex microspheres, individually identifiable by their red and orange fluorescence and a green fluorochrome were used. The use of microspheres with different ratios of red and orange fluorescence made a multiplexed format possible. Many SNPs were analyzed in a single tube. Flow cytometric analysis of the microspheres simultaneously identified both the microsphere type and the fluorescent green signal associated with the SNP genotype.

Polystyrene microspheres (5.5 μm in diameter) with a carboxylated surface and different ratios of red and orange fluorescence were purchased from the Luminex Corp. (Austin, Tex.). All oligonucleotides used for covalent coupling to carboxylated microspheres were synthesized with a 5′ amine group, a C15 or C18 spacer, and 45 nucleotides (Oligos Etc., Bethel, Me. or PE Biosystems, Foster City, Calif.). The 20 nucleotides nearest the 5′ end comprised a common sequence derived from luciferase cDNA (5′-CAG GCC AAG TAA CTT CTT CG-3′) (SEQ ID NO: 59) and were used to determine coupling efficiency to the microspheres by hybridization to a complementary fluoresceinated luciferase probe. The 25 nucleotide cZipCode at the 3′ end were sequences derived from the Mycobacterium tuberculosis genome. This genome was chosen because it was a bacterial genome that had a high GC content. The selected sequences have GC-contents between 56 and 72% and predicted Tₘ values of 61 to 68° C. Reporter oligonucleotides were designed with a 5′ phosphate group and either a 3′ fluorescein or 3′ biotin modification (Oligos Etc., Bethel, Me. or Biosource/Keystone, Camarillo, Calif.). Reporter probe Tₘ ranged from 36-40° C. except for the 8-base reporters which were 18-24° C. Target oligonucleotides had a 25 nucleotide ZipCode sequence at the 5′ end (see Table 1) and an allele-specific sequence at the 3′ end. Allele-specific sequences were designed to possess a Tₘ of 51-56′ C. Target nucleic acids, 150-462 bp in length, were amplified from 10 to 20 nanograms of genomic DNA (CEPH DNA was obtained from Coriell Cell Repositories, Camden, N.J.) using 1.5 units of AmpliTaq Gold (Applied Biosystems, Foster City, Calif.), 400 μM dNTPs, 200 μM forward PCR primer and 200 μM reverse PCR primer in 1× PCR buffer I (Applied Biosystems, Foster City, Calif.). Typically, 30 μl reactions were carried out in PE Biosystems 9700 thermocyclers for 10 minutes at 95° C., followed by 40 three-temperature amplification cycles holding at 94° C., 60° C. and 72° C. for 30 seconds each and ending with an additional 5 minute extension at 72° C. Samples were held at 4° C. following the reaction.

A. Coupling of Oligonucleotides to Microspheres

Carboxylated microspheres (2.5×10⁶ microspheres in 62 μl 0.1 M 2-[N-morpholino] ethanesulfonic acid (MES) (Sigma, St. Louis, Mo.)) were combined with amine-modified oligonucleotide (5 nmoles in 25 μl 0.1 M MES). At three separate times 0.3 mg of 1-ethyl-3-(3-dimethylaminopropyl) carbodilmide hydrochloride (EDC) (Pierce, Rockford, Ill.) was added to the microsphere mixture; at the beginning of the incubation, and then after two 20 min periods. The reaction was occasionally mixed and sonicated during the 60-min room temperature incubation to keep the microspheres unclumped and in suspension. After coupling, the microspheres were washed in 1 ml phosphate buffered saline containing 0.02% Tween 20 (Sigma, St. Louis, Mo.) and then in 150 μl 10 mM tris [hydroxymethyll aminomethane hydrochloride/1 mM ethylenediamine-tetraacetic acid pH 8.0 (TE). The microspheres were resuspended in 200 μl TE for storage at 4° C.

To assess the number of oligos covalently coupled to the microspheres, hybridizations were performed using 10,000 coupled microspheres and 3 picomoles of fluoresceinated oligo complementary to the 20 nucleotides of luciferase sequence on the 5′end of each cZipCode oligo. Hybridization was conducted in 3.3× SSC for 30 minutes at 45° C. following a 2 minute 96° C. denaturation. Microspheres were washed with 200 μl 2× SSC containing 0.02% Tween 20, resuspended in 300 μl 2× SSC containing 0.02% Tween 20 and analyzed by flow cytometry.

B. Specificity of ZipCode Binding

A set of 58 target oligonucleotides was designed and synthesized. Each oligo possessed a common allele-specific 3′ portion, but each contained its own unique 5′ ZipCode sequence. Each taget oligonucleotide was used separately in the standard OLA reaction to assay the common genomic target nucleic acid. The resulting set of 58 fluorescently-labeled OLA products were then used individually in the standard hybridization reaction in the presence of 58 microsphere types, each bearing a different complementary ZipCode. After flow cytometric analysis, ZipCodes which hybridized to multiple types of microspheres were discarded. Replacement ZipCodes were designed, new capture probes were synthesized and the modified set of 58 probes were retested. The number of oligonucleotides coupled to the microsphere was estimated by converting the mean fluorescence intensity to MESF values. Average cZipCode couplings varied from 100,000 to about 1,000,000 MESF per microsphere. Only microspheres that had average couplings ≧100,000 MESF per microsphere were used. ZipCodes were validated for specificity of hybridization by incubating a fluoresceinated oligonucleotide (with a given ZipCode sequence) with a multiplexed set of 58 cZipCode-coupled microspheres (only one microsphere type out of the set of 58 contained a perfectly complementary sequence). Cross-hybridization (or non-hybridization) of ZipCodes was infrequent but when encountered, the sequence was removed from the selection of ZipCodes and replaced with another non-cross hybridizing sequence. Five ZipCode sequences were replaced due to cross reactivity and 2 ZipCode sequences that were at first only weakly reactive showed specific hybridization upon retesting (and were therefore retained). One completely unreactive ZipCode was discarded. A second round of hybridizations demonstrated that, under our assay conditions, each of the 58 ZipCode sequences hybridized to only one of the 58 microsphere-attached, cZipCode sequences. The optimized sequences for ZipCodes are shown in Table 1. We have found no differences in genotyping ability when an SNP was analyzed using different ZipCode sequences.

C. Oligonucleotide Ligation Reaction

Target nucleic acids (double-stranded PCR products, 150-450 base pairs in length) were used at 3-20 ng. Acceptable green fluorescent signals were observed throughout this concentration range. Target oligonucleotides were used at 10 nM and the target oligonucleotide:reporter ratio was 1:50. Reactions were carried out in 10 μl ligase buffer which included the following: 0.1 pmoles target oligonucleotide, 5 picomoles reporter oligonucleotide, 3-20 ng dsDNA target nucleic acid (as determined by Picogreen™ staining, Molecular Probes, Eugene, Oreg.), and 10 U Taq DNA ligase. Incubations were carried out in PE Biosystems 9700 thermocyclers by heating to 96° C. for 2 minutes, followed by 30 cycles of a two-step reaction (denaturation at 94° C. for 15 seconds followed by ligation at 35-37° C. for 1 minute). Samples were held at 4° C. when the cycles were complete. If the 3′ base on the target oligonucleotide is complementary to the target SNP; the fluoresceinated reporter will be ligated to the capture probe. Taq DNA ligase was used at 10 units per 10 μl ligation reaction because, at high concentrations of the enzyme (20 to 80 units ligase per 10 μl reaction volume), the rate of misligation (signal from a capture probe with a mismatched terminal 3′ base) was increased 2 to 7-fold.

D. Hybridization of Target Oligonucleotide/Reporter Probes to Microspheres After OLA

Hybridization of target oligonucleotide/reporter molecules to cZipCode-coupled microspheres was conducted using high salt (750 mM NaCI), small incubation volumes (10-13 μl), and a minimum of 2 hours incubation. cZipCode-coupled microspheres (5,000 to 10,000 of each microsphere type) were added to each ligation reaction. The salt concentration was adjusted to 750 mM NaCl by adding a small volume of 5 M NaCl. The mixture was heated to 96° C. for 2 minutes in a PE Biosystems 9700 thermocycler and then incubated at 45° C. from 2 hours to overnight. Microspheres were washed with 200 μl 2× SSC containing 0.02% Tween 20. When biotinylated reporter probes were used, 5-0 μl of avidin-FITC (Becton Dickinson, San Jose, Calif.) were added to washed, hybridized microspheres resuspended in 30 μl 2× SSC/0.02% Tween 20. The microspheres were incubated for 15 minutes at room temperature and then washed. All microsphere suspensions were resuspended in 300 μl 2× SSC containing 0.02% Tween 20 just prior to flow cytometric analysis. After OLA, the target oligonucleotides, with or without the attached reporter probe, were each hybridized to a specific fluorescent microsphere through the 25-base cZipCode sequence chemically coupled to the microsphere. Microspheres with different ratios of red and orange fluorescence, each bearing a different cZipCode, were multiplexed to analyze several SNPs per tube.

A potential source of background fluorescence was the formation of ‘sandwich’ complexes, non-ligated, ZipCode-hybridized, target oligonucleotide-target nucleic acid-reporter complexes. The background fluorescence contributed by sandwich formation was determined in the absence of ligase. Incubating the microsphere suspension at 45° C. for a minimum of 15 minutes just prior to flow cytometric analysis minimized this background fluorescence (presumably by loss of the short non-ligated reporter molecule from the complex without disturbing the ZipCode hybridization).

E. Flow Cytometric Analysis and MESF Conversions

Microsphere fluorescence was measured using a FACSCalibur flow cytometer (Becton Dickinson, San Jose, Calif.) equipped with Luminex Lab MAP hardware and software (Luminex Corp., Austin, Tex.). All green fluorescence measurements were converted to molecules of equivalent soluble fluorochrome (MESF) using Quantum Fluorescence Kit for MESF units of FITC calibration particles and QuickCal software (all obtained from Sigma, St. Louis, Mo.). Green fluorescence contributed by the microspheres alone were subtracted from all data points. In the experiments described in this paper, both SNP alleles were assayed using the same ZipCode. Hence, alleles were assayed using the same microsphere type in separate tubes. Different alleles for a given SNP in the same tube have also been assayed using unique microsphere types with different ZipCodes.

Conversion of raw data from mean fluorescence intensity to MESF offers several advantages. These advantages include the use of a standard fluorescence unit, the ability to compare data between experiments, the ability to compare data between instruments, and normalization of signal variability in an instrument over time (due to laser power shifts or PMT decline).

F. OLA with Short Degenerate Oligonucleotide Reporter Probes

The OLA reaction was performed with very short reporter oligonucleotides to explore the feasibility of synthesizing a set of all possible reporter sequences that would be needed to analyze SNPs in a high throughput mode. An 8-base sequence that contained either 0 or 2 degenerate sites was used in one set of experiments to minimize the cost of a multiplexing reaction. Thus, a short 8-base oligonucleotide (5′-CTAAGTTA-3′) that constituted a 6+2 mer (an 8-base sequence containing 6 defined and 2 degenerate positions) was designed for SNP and used in the standard OLA reaction. The 8-base reporter probe successfully identified the GG homozygous target DNA. The signal intensity from the 8-base reporter was 65% of that observed with an 18-base reporter. Two 6+2 degenerate reporter oligonucleotides were tested; each contained two degenerate sites, one at positions 3 and 6 and the other at positions 4 and 5. To compensate for the effective 16-fold reduction in concentration of the correct, 5′-CTAAGTTA-3′ sequence, the degenerate reporters were used at 16-fold higher concentrations than the non-degenerate sequence. Both degenerate oligonucleotides correctly identified the SNP genotype of the target DNA.

G. Multiplexed Genotyping of 7 DNA Samples for 9 SNPs

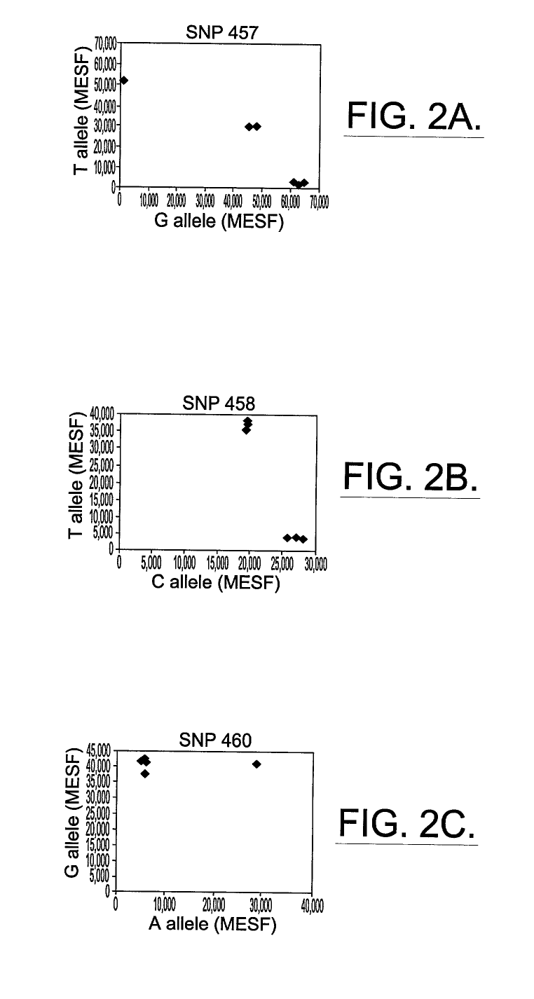

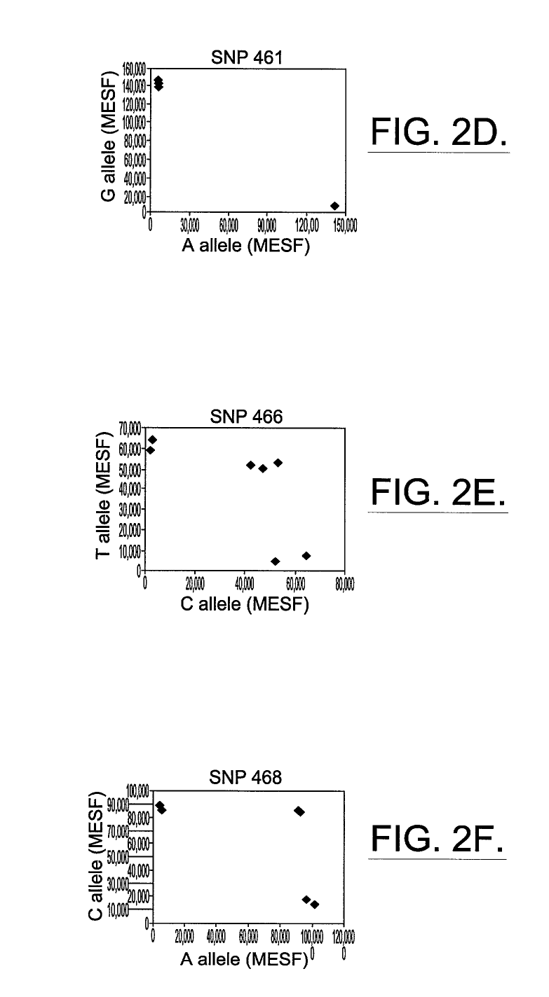

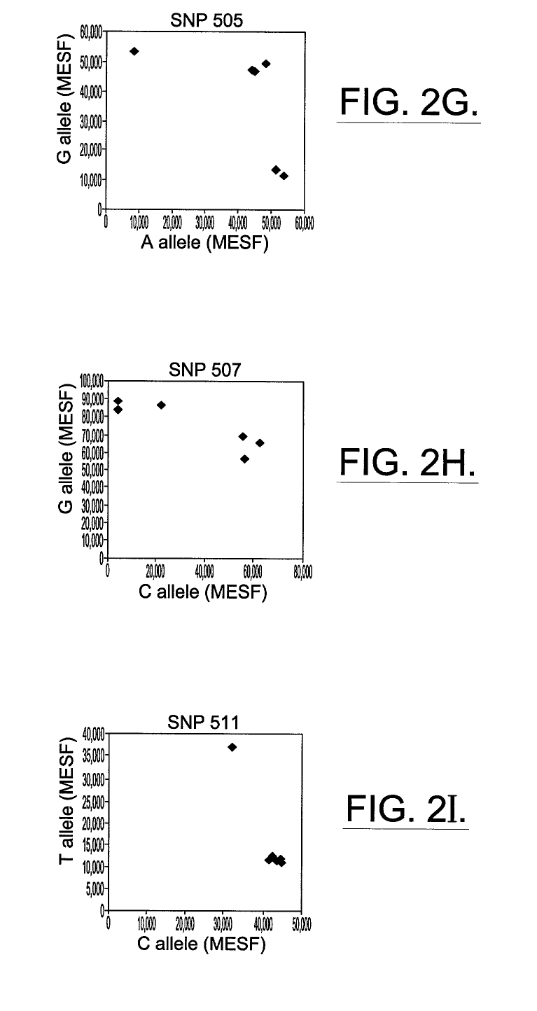

FIG. 2 shows the multiplexed genotyping results from 7 DNA samples for 9 SNPs located near the Apo E locus on chromosome 19 by OLA with a flow cytometric readout. The genomic DNA samples were made available through The Centre d'Etude du Polymorphisme Humain (CEPH) reference panel (http:Hwww.cephb.fr/). In this experiment, each OLA reaction included a pooled mixture of nine target oligonucleotides and reporter probes plus the nine target DNA samples. The different alleles for a given locus were tested in two separate reaction volumes. Each reacted mixture was hybridized to nine microsphere sets in a single step. The 18 possible genotypic analyses for a given individual were therefore conducted in two wells (tubes). ZipCodes 1, 2, 4, 5, 10, 14, 44, 46, and 49 (Table 1) were used for SNPs 457, 458, 460, 461, 466, 468, 505, 507, and 511, respectively. Biotinylated reporters and avidin-FITC were used in this experiment. Table 2 shows zip codes, target oligonucleotide sequences, reporter sequences, and PCR primers used in this experiment.

Homozygous and heterozygous genotypes were readily identified. The microsphere-based SNP analysis agreed with genotyping by direct sequencing in all cases with one interesting note. One individual who was determined to be C homozygous for SNP 466, was found by sequencing analysis to be heterozygous for a third allele (CG). Since the G allele was not included in the experimental design for SNP 466, the individual appeared to be CC in our analysis. In the case of heterozygous targets, fluorescent signal is seen from capture probes for both alleles. The signal intensity of heterozygous patients is, in some cases, slightly less than for homozygous DNA samples. This may be related to relative target probe concentrations which, for heterozygotes, would be half that of homozygotes. Gentotyping results from multiplexed experiments (9 microsphere types per tube) were identical to uniplexed experiments (one microsphere type per tube).

### EXAMPLE 2

A Microsphere-Based Assay for Single Nucleotide Polymorphism Analysis Using Single Base Chain Extension

A rapid, high throughput readout for single nucleotide polymorphism (SNP) analysis was developed employing single base chain extension and cytometric analysis of an array of differentially fluorescent microspheres. The array of fluorescent microspheres are coupled with uniquely identifying sequences, termed complementary ZipCodes (cZipCodes), which allow for multiplexing possibilities. For a given assay, querying a polymorphic base involves extending an oligonucleotide containing both a ZipCode and an SNP-specific sequence with a DNA polymerase and a pair of fluoresceinated dideoxynucleotides. To capture the reaction products for analysis, the ZipCode portion of the oligonucleotide hybridizes with its complementary ZipCodes (cZipCodes) on the microsphere. Flow cytometry is used for microsphere decoding and SNP typing by detecting the fluorescein label captured on the microspheres. In addition to multiplexing capability, the ZipCode system allows multiple sets of SNPs to be analyzed by a limited set of cZipCode attached microspheres. A standard set of noncross reactive ZipCodes was established experimentally as described above, and the accuracy of the system was validated by comparison with genotypes determined by other technologies.

As used in the present example, AmpliTaq, AmpliTaq Gold and AmpliTaq FS (catalog number: 361390) DNA polymerase were purchased from Perkin-Elmer Applied Biosystems (Foster City, Calif.). KlenTaq was obtained from Ab Peptides, Inc. (St. Louis, Mo.). PicoGreen for double strand DNA quantification was purchased from Molecular Probes (Eugene, Oreg.). Shrimp alkaline phosphatase (SAP) and Exonuclease I (Exo 1) were obtained from Amersham Pharmacia (Cleveland, Ohio). Fluorescence labeled dideoxynucleotide triphosphates (ddNTPs) were obtained from NEN Life Science Products, Inc. (Boston, Mass.). Unlabeled ddNTPs were from Amersham Pharmacia (Cleveland, Ohio). Unmodified oligonucleotides were purchased from Keystone Biosource (Camarillo, Calif.). CEPH DNAs (NA07435, NA07445, NA10848, NA10849, NA07038A, NA06987A and NA10846) are ordered from Coriell Cell Repositories (Camden, N.J.). Oligonucleotides with 5′ amino group were ordered from Oligo Etc. (Wilsonville, Oreg.) or from Perkin-Elmer Applied Biosystems.

2-[N-Morpholino]ethanesulfonic acid (MES) and 1-Ethyl-3-(3-Dimethylaminopropyl)carbodiimide Hydrochloride (EDC) were purchased from Sigma (St. Louis, Ill.) and Pierce (Rockford, Ill.), respectively. DNA polymerase was cloned from Thermatoga neapolitana (Tne) (see U.S. Pat. Nos. 5,912,155; 5,939,301; and 5,948,614, which are incorporated herein by reference) and expressed in Escherichia coli (E. coli). The Klenow fragment (TneK), lacking the 5′ to 3′ exonuclease was used for SBCE reactions under the same assay conditions for AmpliTaq. Details of both the cloning and expression of Tne, TneK and TneK FS and their performance in SBCE will be submitted elsewhere. Carboxylated fluorescent polystyrene microspheres were purchased from the Luminex Corp. (Austin, Tex.).

A. Coupling of Oligonucleotides to Microspheres

As described in the previous example, capture oligonucleotides with a 5′ amino group were coupled to the carboxyl group on the surface of the microspheres. In these oligonucleotides, a carbon spacer (C15-18) was synthesized adjacent to the 5′ amino group to reduce the potential interference of the oligonucleotide hybridization by the microspheres and the luciferase sequence described above was used to monitor the coupling efficiency of the oligonucleotides to the microspheres. A 25-base complementary ZipCode sequence (named cZipCode, see Table 1) was arbitrarily selected from the Mycobacterium tuberculosis genome and validated experimentally (as above). Carboxylated microspheres (2.5×10⁶) in 62 μl of 0.1 M MES buffer were mixed with 5 nmoles of oligonucleotides in 0.1 M MES (6.25 μl). Freshly made 30 mg/ml EDC (10 μl) was added to the microspheres/oligo mixture and incubated at RT for 20 min. Two additional rounds of 10 μl EDC were added at intervals of twenty minutes. The reaction mixture was mixed occasionally and sonicated during incubation to assure microsphere separation and suspension. After a total incubation period of 60 min, the microspheres were washed twice with 1 ml of Phosphate Buffered Saline (PBS) plus 0.02% Tween 20, rinsed with 150 μl of TE [Tris[hydroxymethyl]aminomethane hydrochloride (10 mM)/1 mM Ethylenediamine-tetraacetic acid (pH 8.0)], resuspended in 250 μl TE and stored at 4° C. The number of the oligonucleotides coupled to the microspheres was assessed by hybridizing a fluorescent-labeled sequence that is complementary to the SeqLuc sequence. Microspheres with a minimum MESF value of 100,000 were used in SBCE experiments.

B. PCR Amplification

PCR reactions were performed in a 96-well plate on a GeneAmp 3700 thermal cycler (Perkin-Elmer). A typical 30 μl reaction mixture contained 10 mM Tris-HCl (pH 8.3), 50 mM KCl, 1.5 mM M_{g}Cl₂, 0.1 mM dNTPs, 0.2 μM of each primer, AmpliTaq Gold DNA polymerase (1.5 units) and 20 ng genomic DNA. The reaction mixture was held at 95° C. for 10 min to activate the DNA polymerase and the amplification was carried out for 9 cycles at 94° C. for 10 sec, 61° C. for 45 sec and 72° C. for 90 sec, 9 cycles at 94° C. for 10 sec, 56° C. for 45 sec and 72° C. for 90 sec and another 25 cycles at 94° C. for 10 sec, 61° C. for 45 sec and 72° C. for 90 sec. After another 5-min extension at 72° C., the reaction mixture was held at 4° C.

C. Quantitation of PCR Products, Primer and dNTP Degradation

PCR products were quantified using the PicoGreen binding assay according to the manufacturer's instructions (Molecular Probes, Eugene Oreg.). The fluorescence intensity was measured using a CytoFluor MultiWell Plate Reader Series 4000 (PE Blosystems) and the quantity was calculated against DNA standards with known quantities. To degrade the PCR primers and dNTPs, 1 unit of SAP and 2 units of E. coli exonuclease I were added directly to 10 μl of PCR reaction mixture. The reaction was incubated at 37° C. for 30 min, then at 99° C. for 15 min for enzyme inactivation. Some PCR products were cleaned with the Qiagen Qiaquick kit (Qiagen, Valencia, Calif.).

D. SBCE Reactions

To either single or pooled PCR products (10-20 ng each), a SBCE reaction mixture was added to a total volume of 10 μl. The mixture consists of 80 mM Tris-HCl (pH 9.0), 2 mM MgCl₂, 100 nM of target oligonucleotide, 3 units of AmpliTaq FS (Perkin-Elmer), 10 μM of each allele specific FITC-labeled ddNTP and 30 μM of unlabeled other three dNTPs. The reaction mixture was incubated at 96° C. for 2 min followed by 30 cycles of 94° C. 30 sec, 55° C. for 30 see and 72° C. for 30 sec. Reactions were held at 4° C. prior to the addition of microspheres.

As unmodified double-stranded PCR product was used as template in our system, several thermostable DNA polymerases were evaluated under thermo-cycling conditions for efficacy of fluorescein (FITC) labeled ddNTP incorporation. One PCR product containing a T/C polymorphism (SNP 18) was analyzed with both sense and anti-sense capture oligonucleotides for T, C, A and G incorporation, respectively. Allele specific incorporation of the correct base was tested using two homozygous (CC and TT) and a heterozygous (CT) PCR fragments generated from genomic DNA samples. AmpliTaq FS generated the highest signals and a ratio between positive signals and non-specific incorporation (noise) of greater than 100-fold for both ddATP-FITC and ddGTP-FITC incorporation across the three genotypic possibilities. AmpliTaq, KlenTaq and TneK produced much weaker signals and a significantly reduced signal-noise ratio. Similar results were obtained for the incorporation of T and C bases by both AmpliTaq FS and the other DNA polymerases tested. These data clearly demonstrate that a microsphere-based SBCE assay system works well and that AmpliTaq FS is an appropriate choice for incorporating fluorescein-labeled ddNTPs under the conditions used. AmpliTaq FS, an F667Y version of Taq Pol 1, does not discriminate between deoxy- and dideoxynucleotides.

E. Hybridization of SBCE Reaction Mixture to the Microsphere

After the SBCE reactions, each of the allele specific extension products was captured by its corresponding microspheres containing the cZipCode complementary sequence. A pool of different microspheres was treated with bovine serum albumin (BSA) at 1 mg/ml for 30-60 min at 37° C. and then concentrated by centrifugation at 3000 g for 5 min. Approximately 1200 of each fluorescent microsphere were added to the 10 μl SBCE reaction mixture for a final volume of 15 μl. The concentrations of NaCl and EDTA were adjusted to 1 M and 20 mM respectively. The mixture was incubated at 40° C. for 2 h or more. Microspheres were washed by the addition of 200 μl of 2× SSC (1× SSC is 8.77 g of NaCl plus 4.41 g of sodium citrate per liter (pH 7.0))-0.02% Tween 20 at room temperature (RT). After centrifugation at 1100× g for 6 min, the pelleted microspheres were resuspended in 250 μl 2× SSC 0.02% Tween 20 for flow cytomertry analysis.

F. Flow Cytometric Analysis and MESF Conversions

Microsphere fluorescence was measured using a FACSCalibur flow cytometer (Becton Dickinson) equipped with Luminex Lab MAP hardware and software (Luminex Corp, Austin, Tex.) as described above.

G. Analysis of SNPs in Multiplexed Reactions

A primary advantage of the LumineX™ fluorescent microsphere technology is the capacity for conducting multiple biological reactions simultaneously in a single reaction vessel (i.e. well). By synthesizing stocks of unique pairings between microspheres and cZipCodes (DNA sequences), each fluorescent microsphere becomes the address for a single SNP. Each SNP then simply requires an assigned ZipCode encoded in the capture oligonucleotide to permit multiplexing. To test this hypothesis, four polymorphisms with T to C changes were assayed in multiplex reactions. PCR products generated from either homozygous (CC or TT) or heterozygous (CT) genomic DNAs were pooled separately and four capture oligonucleotides were mixed as primers in SBCE reactions. All of the four SNPs were genotyped correctly based on signal strength as measured by mean equivalence soluble fluorochrome (MESF values). The background MESF values were only a few percent of the specific signals. It is interesting to note that the signals for both the A and the G reactions were close to the background in the absence of specific PCR template (TT) for the two SNPs. This indicates the absence of hybridization of those capture oligonucleotides and the other unrelated DNA templates. The results were nearly identical for the T and C reactions using the capture oligos for the opposite strand.

H. Optimization of the Microsphere-Based SBCE Reactions

When the need arises for large numbers of SNPs to be assayed in thousands of DNA samples, a reliable robust assay with minimal reagent cost will be essential. Therefore, a large number of experiments were performed to optimize the reaction conditions. A titration curve of AmpliTaq FS for SNP 18 in a multiplex reaction of four SNPs (the same SNPs were used as described in the previous multiplex experiment) was generated. A homozygous mixture of PCR products (CC) of the four SNPs was used as template and was assayed for alleles A and G with the anti-sense capture oligonucleotide. The specific signal of the G reaction was very high and the A reaction remained low. Similar results were obtained for the other three SNPs. There was no significant increase of signal between 0.5 to 8 units of the DNA polymerase used.

The signal strengths of SNP18 at various concentrations of ddNTP-FITC was analyzed. The reactions were performed in the presence of three other SNPs, and the results for the four SNPs were nearly identical. PCR product amplified from homozygous (CC) DNA sample was used as template and the anti-sense capture oligonucleotide was used as primer. Specific incorporation of ddGTP-FITC was found to generate strong signal while the signal for the A reaction was near the background level. Signals were found to remain constant as the concentration of FITC-ddNTP was reduced from 10 μM to I μM. A near linear increase of specific signal (G reaction) was observed when the ddNTP was at a much lower concentration (from 20 to 750 nM) in the SBCE reaction.

A key component of the microsphere-based SBCE system is the target oligonucleotide, which is used both as the primer for the base incorporation and as the anchor for the resultant SBCE products to be hybridized to the appropriate microsphere. Various concentrations of the target oligonucleotide were analyzed under the standard conditions. No significant difference was observed between 10 to 100 nM. The signals were found to be significantly reduced when the target oligonucleotide concentration increased to 125 nM.

The level of PCR amplification varies and is dependent, among other factors, upon primers and template sequences. Thus, the sensitivity and tolerance of the microsphere-SBCE assay were tested with various amounts of PCR products under the standard conditions. In this experiment, PCR product amplified from homozygous (CC) genomic DNA was used and assayed for either the specific incorporation of a C nucleotide or the non-specific incorporation of a T nucleotide. While the non-specific T incorporation remained near zero, the signal from the C reaction was found to increase with increasing quantity of PCR product (up to 40 ng). The specific signals were proportional to the amount of PCR products used, up to 2.5 ng. The correct genotypes were generated in the presence of as little as 0.5 ng of PCR product, where the MESF values for the C and T reactions were 4400 and 200 respectively. Thus the assay system is fairly sensitive and can tolerate up to an 80-fold variation of template material.

I. Validation of the Microsphere-Based SBCE Assays

It is well known that one allelic variant of the apolipoprotein, APOE4, is a significant susceptibility allele or risk factor for younger age of onset of Alzheimer disease. Over a hundred SNPs have been developed around the APOE gene for association studies (Lai, E., Riley, J., Purvis, I. & Roses, A. A 4-MB high-density single nucleotide polymorphism-based map around human APOE. Genomics 54, 31-38 (1998)). These SNPs were identified by DNA sequencing of amplicons from the seven CEPH DNAs and therefore, nearly all of the genotypes for those SNPs are available (Id.). A total of 58 SNPs were randomly selected from this set and SBCE assays were developed. Each of the SNPs utilized a unique ZipCode sequence (Table 1).

A typical set of these experiments for analyzing these 58 SNPs is described below. Each of these SNPs was amplified individually across the seven CEPH DNAs and PCR products were quantified using PicoGreen assays. Equal amounts of PCR products were pooled for 12 SNPs from each of the CEPH DNAs and were assayed for all four bases. Of the total 58 SNPs, 54 SNPs were converted to the assay format successfully in the first pass. Only two of these SNPs failed completely in the assay and showed no incorporation of any of the four nucleotides. Another two SNPs (462, 492) generated accurate genotypes but had very low specific signal (1300 to 2200). However, the level of background noise was less than 500 MESF. In these experiments, any specific signal below 3000 MESF was arbitrarily deemed a failure. The target oligos for assaying the other strand were designed and all four SNPs were successfully rescued.

Based on the signal intensity of each of the four alleles in the seven CEPH DNAs for SNP503, the genotype can be easily read as GG, AG, GG, AG, GG, AG. Because of the dramatic difference between the signal and noise, all of the remaining 77 genotypes could be easily determined as well. The 12 SNPs represent several different types of base substitutions (AG, AT, CG, CT and GT). All of the five examined can be analyzed in a simple multiplex reaction by assaying the four bases.

A total of 180 genotypes determined by SBCE from 54 SNPs (21 SNPs assayed in 7 DNAs and 33 SNPs in one DNA) were compared to their known genotypes as determined by either DNA sequencing or TaqMan analysis. All of the 180 genotypes generated from our assays were proven to be correct.

J. Multiplex Reactions

PCR products from 12 SNPs were analyzed using DNA from seven CEPH DNAs. The A, C, G and T assays were performed with 10 ng of PCR products for each SNP in a 12 μl reaction to accommodate the large volume of PCR products. The reagents were increased proportionally to what was described above. A mixture of 12 different microspheres was pre-treated with 1 mg/ml BSA for 45 min and hybridization was left overnight at 40° C. before the flow cytometric analysis. The average MESF values of about 120 microspheres are shown.

To test the limit of higher multiplexing capacity, PCR products from 52 SNPs were pooled from a DNA sample that had been analyzed in our system. All of the 52 genotypes determined from this experiment were found to be the same as in the 12 SNP multiplex reactions. Therefore all of the 52 genotypes could be correctly determined in a single multiplex reaction.

### EXAMPLE 3

A Microsphere-Based Assay for Single Nucleotide Polymorphism Using Minisequencing (Allelle-Specific Polymerization Reaction)

The SBCE methods described above were modified to perform an allelle-specific polmerization reaction. Thus, instead of using a labeled chain terminating dideoxynucleotide, a labeled deoxynucleotide was used. As for the SBCE reaction, the allelle-specific polymerization reaction was multiplexed by using more than one labeled deoxynucleotide. The results show that the genotype was properly predicted using allele-specific polymerization as the identification reaction in a single tube or multiple tube format.

### EXAMPLE 4

A Microsphere-Based Analysis of Microbial Contamination Using Single Base Chain Extension (SBCE)

A. DNA Extraction from Bacteria

The procedure removes the proteins and cell debris that could potentially inhibit SBCE reaction. The following reagents were used: bacterial colonies on nutrient agar, sterile/DNAse-free water, 5 mg/mL lysozyme, 3.75 mg/mL lysostaphin, TE buffer (10 mM Tris; 1 mM EDTA), 0.25M EDTA, 1M DTT, 20 mg/mL Proteinase K, 10% SDS, Perkin Elmer's PrepMan© Reagent.

Approximately ¼ of a large loopful of colonies from a nutrient agar plate was resuspended in 245 uL TE buffer in a sterile microcentrifuge tube. Lysozyme (5 μl) was added to the cell suspension, and the suspension was mixed gently by tapping. Lysostaphin (5 μl) was used as the lysozyme when extracting DNA from Staphylococcus.

The cell suspension was then incubated for 45 minutes at 56 C. The following were added to the cell suspension: 196.2 uL TE, 5.0 uL DTT, 20.0 uL EDTA, 25.0 uL SDS, 8.8 uL Proteinase K. The suspension was mixed gently by tapping and subsequently incubated for 1 hour at 37 C. The PrepMan reagent was vortexed briefly to resuspend the contents, and 500 uL of PrepMan reagent was added to the cell suspension. The suspension was incubate for 30 minutes at 56 C, and then vortexed for 10 seconds. The cell suspension was then incubated in a boiling water bath for 8 minutes to lyse the cells. The lysed cell suspension was vortexed briefly and centrifuged in a microcentrifuge for 2 minutes at 11,000 RPM to pellet the cell debris. The DNA was diluted 1:10 by adding 100 uL of the supernatant to 900 uL sterile water, which can optionally be stored 4 C for up to one week. Prior to the PCR reaction, the DNA is further diluted to make a 1:250 dilution by adding 100 uL of the 1:10 dilution to 2.5 mL sterile water. Ten uL of this 1:250 dilution will be used in the PCR reaction.

As an alternative method of DNA extraction a modification of the protocol of K. Boye et al. (1999 Microbiol. Res 154: 23-26) can be used as follows: one bacteria colony (2-3 mm diameter) was picked from a Petri-Dish and suspended in 500 μl water. After incubation at 95 C for 15 min, the samples were centrifuged at 15000 g for 5 min. The pellet was resuspended in 200 μl of 5% Chelex-100 resin (Bio-Rad) by vigorous shaking. The Chelex-100 resin and cell debris was pelleted by 1 min of centrifugation. Typically, 5 μl of crude DNA can be used for the PCR amplification.

B. PCR Amplification of the Bacterial DNA for 16S Sequencing

This method describes the procedure for performing the polymerase chain reaction (PCR) to amplify the 16s region of bacteria genomes in preparation for the SBCE reaction. The following reagents were used: AmpliTaq Gold DNA polymerase, Perkin Elmer 10× PCR buffer 10 mM dNTP mix (2.5 mM each dNTP), PCR primers (27F-5′-AgAgTTTgATCMTggCTCAg-3′ and 1525R-5′-AAggAggTgWTCCARCC-3′), sterile/DNAse-free water, 10× TBE buffer, agarose, Molecular Probes' SYBR green nucleic acid gel stain, gel loading dye, molecular weight marker.

PCR Reaction Mixtures were prepared as follows: All reagents were thawed at room temperature then store in ice until use. AmpliTaq Gold was diluted 1:5 in sterile water. Primer 27F and primer 1525R were diluted 1:10 in sterile water. A master mix was prepared using the specified volumes per PCR reaction (5.0 uL PCR buffer, 4.0 uL dNTP mixture, 2.0 uL 1:5 diluted AmpliTaq Gold, 2.2 uL 1:10 diluted primer 27F, 2.4 uL 1:10 diluted primer 1525R, 24.4 uL sterile/DNAse-free water).

Forty uL volumes of the master mix were pipetted into 200 uL PCR tubes and 10 uL of the 1:250 diluted DNA template was added. PCR Thermalcyling was performed using a GeneAmp 9600 thermalcycler: 1 cycle at 95 C for 10 minutes; 35 cycles of 94 C for 30 seconds, 56 C for 45 seconds, and 72 C for 90 seconds; 1 cycle of 72 C for 5 minutes, and one cycle at 4 C. The thermalcycler will run the method for approximately 3 hours.

The PCR products were subsequently detected using gel electrophoresis. The agarose gel was prepared according to methods well known in the art. Ten uL of PCR product was combined with 2 uL of gel loading dye, and 10 uL of the sample were loaded into a well in the gel. Eight uL of the molecular weight marker was loaded into a separate well, and the gel was run under standard conditions. The gel was subsequently stained in approximately 50 mL TBE buffer containing 8 uL SYBR Green nucleic acid gel. The gel was then viewed in a UV light to ensure that the size of the PCR product was approximately 1500 bp by comparing the band to the control bands in the molecular weight marker.

In an alternative protocol of PCR amplification, the PCR reaction conditions were 10 mM Tris-HCl (pH 8.3), 50 mM KCl, 1.5 mM MgCl₂, 0.001% gelatin, 0.1 mM each of the four dNTPs, 1 unit AmpliTag GOLD DNA polymerase (Perkin-Elmer) and 50 pmol of each primer (27f/1525r or 66f/1392r (R. Ghozzi et al, 1999, J. Clinical Microbiology 37: 3374-3379)). in a total volume of 50 μl. An initial detaturing step of 95° C. for 10 min was followed by 30 cycles of amplification (1 min at 94° C., 1 min at 55° C., and 2 min at 72° C.). Ten μl of the PCR product was analysed on an agarose gel.

C. Purifying DNA from PCR Product

To purify DNA from PCR product, Qiagen's QiaQuick PCR Purification Kit, containing Buffer PB, Buffer PE, QiaQuick columns, and 2 mL collection tubes, was used. Five volumes of Buffer PB was mixed with 1 volume of PCR reaction mixture in a microcentrifuge tube. A QiaQuick spin column was placed in a 2 mL collection tube and the entire sample was applied to the QiaQuick column. The column was centrifuged for 60 seconds at 11,000 RPM. The flow-through was discarded, and the column was placed in the same 2 mL tube.

To wash the DNA, 0.75 mL Buffer PE was added to the column. The column was centrifuged for 60 seconds at 11,000 RPM. The flow-through was again discarded, and the column placed in the same 2 mL tube. The column was then centrifuged for 60 seconds at 11,000 RPM to remove any residual ethanol. The flow-through was once again discarded. The column was then placed in a clean micrcentrifuge tube.

To rehydrate the DNA, 30 uL sterile/DNAse-free water were added to the center of the QiaQuick membrane. The DNA was allowed to rehydrate for not less than 1 minute at room temperature, and the column was subsequently centrifuged for 60 seconds at 11,000 RPM to elute the DNA from the column.

The DNA was then quantified using absorbance spectroscopy using a Milton Roy Spectronic 1201.

D. Luminex Bead Protocol

The following reagents were used in the single base chain extension/bead protocol: shrimp alkaline phosphatase (SAP), Exonuclease I, 5 uM probe+zipcode, 5× SBCE buffer, AmpliTaq FS, 10 uM ddNTPs, 10 uM R6G-ddNTPs, 250 nM control oligonucleotides, 5M NaCl, 130 mM EDTA, and 22 separate bead sets (1000 of each) (0.1 uL of 10K/uL).

To each DNA suspension, 1 uL SAP and 0.2 uL ExoI were added per 10 uL volume, or, alternatively, a mixture of 20 ul of water and 25.5 ul (˜400 ng) from the PCR products was dispensed into Whatman plate for ExoI/SAP digestion using 5.5 ul of ExoI/SAP mixture (50 ul SAP (1 U/ul, USB E70092Y) and 5 ul Exo I (10 u/ul, USB E70073Z)). The clean-up reaction was performed at 37 C for 30 minutes, the enzymes were disabled at 99 C for 30 minutes, and the mixture held at 4 C. Two ul aliquots (˜10-20 ng/rxn) were used for the SBCE reaction.

A master mix for the SBCE reaction was prepared with the following reagents: 0.1 ul 5 uM Probe/ZipCode, 4.0 ul 5× Buffer, 0.2 ul AmpliTaq FS, 2.0 ul 10 uM each cold ddNTP, 2.0 ul 10 uM R6G ddNTP, 1.0 ul 250 nM control oligonucleotide, 0.7 water. Ten uL of the master mix were added to 10 uL of the clean DNA template. Alternatively, 2 ul of the PCR product was transferred to another plate and 10 ul of the following SBCE reaction mixture was added: 25 nM 5 uM probe/ZipCode mix, 1× Amplitaq Buffer, 2.8 units/reaction of Amplitaq FS (12 u/ul), approximately 10 ng/reaction of PCR product, 3 uM cold ddNTP, 1 uM labeled ddNTP (e.g., 10 uM R6G-ddATP, -ddCTP, -ddGTP, or -ddUTP). The plate was then placed in the thermalcycler and the SBCE reaction was run under the following conditions: 1 cycle of 96 C for 2 minutes; 30 cycles of 94 C for 30 seconds, 55 C for 30 seconds, 72 C for 30 seconds; and 1 cycles at 4 C to hold.

Ten ul of the following mix was subsequently added to each well: 3 ul of 5M NaCl, 0.8 ul 0.5M EDTA, 2 ul of bead mix, 4.2 ul water for a total volume of 10 ul and a final concentration of 0.5M NaCl, 13 mM EDTA, and 1,000 beads per reaction. The samples were then mixed gently and placed on a MJ Research thermal cycler (96 C, 2 min: 40 C, 60 min:End).

When biotin is used for labeling rather than R6G-dNTP in the SBCE step, washing was required. To wash the beads, 110 ul of wash solution (1× SSC, 0.02% Tween) was pipetted into each well. Using the reset button, browse the settings for the program. The plates were then centrifuged at 2500 rpm for 5 minutes, and the supernatant was removed. Three washes were performed.

The fluorescence of the beads and label were analyzed using the LX100 plate reader and Luminex software. One hundred events/bead as a standard number of events were counted using about 40 ul of sample and a flow setting of 60 ul/minute.

The results of this study show incorporation of the proper chain-terminating nucleotide for each known contaminant.

Throughout this application, various publications are referenced. These publications are hereby incorporated by reference in their entirety.

While the invention has been described with respect to certain specific embodiments and examples, it will be appreciated that many modifications and changes may be made by those skilled in the art without departing from the spirit of the invention. It is intended, therefore, by the appended claims, to cover all such modification and changes as fall within the true spirit and scope of the invention.

TABLE 1
Zip Code Sequences^{a}

| | ZipCode | | ZipCode | | |
|---|---|---|---|---|---|
| | Designation | DNA Sequence | Designation | DNA Sequence | |
| SEQ ID NO 1 | 1 | GATGATCGACGAGACACTCTCGCCA | 35 | ACGACTGCGAGGTGCGGTAAGCACA | SEQ ID NO 30 |
| SEQ ID NO 2 | 2 | CGGTCGACGAGCTGCCGCGCAAGAT | 36 | GCGATCGCCGGGAGATATACCCAAC | SEQ ID NO 31 |
| SEQ ID NO 3 | 3 | GACATTCGCGATCGCCGCCCGCTTT | 37 | TCGTGCCGGACTCGAGCACCAATAC | SEQ ID NO 32 |
| SEQ ID NO 4 | 4 | CGGTATCGCGACCGCATCCCAATCT | 38 | GCTTTAGCACCGCGATGGCGTA.GAC | SEQ ID NO 33 |
| SEQ ID NO 5 | 5 | GCTCGAAGAGGCGCTACAGATCCTC | 39 | CAGCCGCGGTACTGAATGCGATGCT | SEQ ID NO 34 |
| SEQ ID NO 6 | 6 | CACCGCCAGCTCGGCTTCGAGTTCG | 40 | CCCCGGATAGCTGACGAGCTTACG | SEQ ID NO 35 |
| SEQ ID NO 7 | 7 | CGACTCCCTGTTGTGATGGACCAC | 41 | TCCGGACAGGTTGGGGTGCGTTTGG | SEQ ID NO 36 |
| SEQ ID NO 8 | 8 | CTTTTCCCGTCCGTCATCGCTCAAG | 42 | CGTAGAGCAACGCGATACCCCCGAC | SEQ ID NO 37 |
| SEQ ID NO 9 | 9 | GGCTGGGTCTACAGATCCCCAACTT | 44 | AGCAGCAGTGACAATGCCACCGCCG | SEQ ID NO 38 |
| SEQ ID NO 10 | 10 | GAACCTTTCGCTTCACCGGCCGATC | 46 | TCGCCCGCGGACACCGAGAATTCGA | SEQ ID NO 39 |
| SEQ ID NO 11 | 12 | TTTCGGCACGCGCGGGATCACCATC | 48 | GAGGCAGATCCGTAGGCGGGTGCAT | SEQ ID NO 40 |
| SEQ ID NO 12 | 14 | CTCGGTGGTGCTGACGGTGCAATCC | 49 | GCGATAGCCAGTGCCGCCAATCGTC | SEQ ID NO 41 |
| SEQ ID NO 13 | 15 | TCAACGTGCCAOCGCCGTCCTGGGA | 50 | AGCGGTCACCATGGCCACGAACTGC | SEQ ID NO 42 |
| SEQ ID NO 14 | 16 | GCGAAGGAACTCGACGTGGACGCCG | 51 | TTGCAACAGCAGCCCGACTCGACGG | SEQ ID NO 43 |
| SEQ ID NO 15 | 17 | CGGGGATACCGATCTCGGGCGCACA | 52 | TGACTCCGGCGATACGGGCTCCGAA | SEQ ID NO 44 |
| SEQ ID NO 16 | 18 | GGAGCTTACGCCATCACGATGCGAT | 53 | ACCGGCTACCTGGTATCGGTCCCGA | SEQ ID NO 45 |
| SEQ ID NO 17 | 19 | CGTGGCGGTGCGGAGTTTCCCCGAA | 54 | GAGCGAGCGGGCAAACGCCAGTATC | SEQ ID NO 46 |
| SEQ ID NO 18 | 20 | CGATCCAACGCACTGGCCAAACCTA | 55 | AGTCGAAGTGGGCGGCGTCAGACTC | SEQ ID NO 47 |
| SEQ ID NO 19 | 21 | CTGAATCCTCCAACCGGGTTGTCGA | 56 | CACCACCAGTGCCGCTACCACAACG | SEQ ID NO 48 |
| SEQ ID NO 20 | 22 | TTCGGCGCTGGCGTAAAGCTTTTGG | 57 | CCGTGTTAACGGCGCGACGCAAGGA | SEQ ID NO 49 |
| SEQ ID NO 21 | 23 | GTAA.ATCTCCAGCGGAAGGGTACGG | 58 | GAGTGAACGCAGACTGCAGCGAGGC | SEQ ID NO 50 |
| SEQ ID NO 22 | 24 | CCGGCTTTGAACTGCTCACCGATCT | 59 | CGGCGGTCTTCACGCTCAACAGCAG | SEQ ID NO 51 |
| SEQ ID NO 23 | 27 | ACTACGCAACACCGAACGGATACCC | 60 | GTTGGGCCCGAGCACTGCAAGCACC | SEQ ID NO 52 |
| SEQ ID NO 24 | 28 | GGACCAATGGTCCCATTGACCAGGT | 61 | TCGGCGTACGAGCACCCACACCCAG | SEQ ID NO 53 |
| SEQ ID NO 25 | 29 | CAACGCTGAGCGCGTCACTGACATA | 62 | CCCCAAACGTACCAAGCCCGCGTCG | SEQ ID NO 54 |
| SEQ ID NO 26 | 31 | GAGACAAAGGTCTGCGCCAGCACCA | 63 | ATGGCACCGACGGCTGGCAGACCAC | SEQ ID NO 55 |
| SEQ ID NO 27 | 32 | TGGCCACACTGTCCATTTGCGCGGT | 64 | AGCCGCGAACACCACGATCGACCGG | SEQ ID NO 56 |
| SEQ ID NO 28 | 33 | CCTTGCGACGTGTCAAOTTGGGGTC | 65 | CGCGCGCAGCTGCAGCTTGCTCATG | SEQ ID NO 57 |
| SEQ ID NO 29 | 34 | AGGTTAGGGTCGCGCCAAACTCTCC | 66 | TACCGGCGGCAGCACCAGCGGTAAC | SEQ ID NO 58 |

^{a}Selected from the Mycobacterium tuberculosis genome, all sequences are written 5′ to 3′

TABLE 2
Oligonucleotide Sequences for Multiplexed Genotyping of 7 DNA Samples for 9 SNPs^{a.}

| | | | | | PCR Primers |
|---|---|---|---|---|---|
| SNP^{b} | Allele | Zip Code | Target Oligo Sequence | Reporter Sequence^{d} | Forward/Reverse |
| 457 | G | 1 | AGTGGGTCTCAACCACTATAAAg | CCTCTCTGTGCC | Fwd. ACTGTCATTGCCCTTTCTGTCC |
| | | | (SEQ ID NO:68) | (SEQ ID NO:69) | (SEQ ID NO:70) |
| | | | | | Rev. CACACAGTCATGGTTCCAACACG |
| | | | | | (SEQ ID NO:71) |
| 457 | T | 1 | AGTGGGTCTCAACCACTATAAAt | | |
| | | | (SEQ ID NO:72) | | |
| 458 | C | 2 | GGAGAAAGGCCAGTCCATc | GACGACATGATCC | Fwd. ATTTGACGTGTCCAACGC |
| | | | (SEQ ID NO:73) | (SEQ ID NO:74) | (SEQ ID NO:75) |
| | | | | | Rev. TGGAACTCTGGTTGAAACTG |
| | | | | | (SEQ ID NO:76) |
| 458 | T | 2 | GGAGAAAGGCCAGTCCATt | | |
| | | | (SEQ ID NO:77) | | |
| 460 | A | 4 | ATCTGATTGGCTTTCTGAGGTTTa | GCTGGGTGGGG | Fwd. CCACTGGCTGCTGTTCTGAAAC |
| | | | (SEQ ID NO:78) | (SEQ ID NO:79) | (SEQ ID NO:80) |
| | | | | | Rev. AAGCGACCATCCCCACATCCATTC |
| | | | | | (SEQ ID NO:81) |
| 460 | G | 4 | ATCTGATTGGCTTTCTGAGGTTTg | | |
| | | | (SEQ ID NO:82) | | |
| 461 | A | 5 | CTCATTTGGCCACTCTGCAa | ATTGGACTTGCCC | Fwd. CCACTGGCTGCTGTTCTGAAAC |
| | | | (SEQ ID NO:83) | (SEQ ID NO:84) | (SEQ ID NO:85) |
| | | | | | Rev. AAGCGACCATCCCCACATCCATTC |
| | | | | | (SEQ ID NO:86) |
| 461 | G | 5 | CTCATTTGGCCACTCTGCAg | | |
| | | | (SEQ ID NO:87) | | |
| 466 | C | 10 | CTTATATAGCTGCGCGGGAAc | AAGGTTGTCCTGC | Fwd. AAATGAGACGGTTTGGGGAGCGAG |
| | | | (SEQ ID NO:88) | (SEQ ID NO:89) | (SEQ ID NO:90) |
| | | | | | Rev. GTGACAGAGAATGAGTTTGCGATG |
| | | | | | (SEQ ID NO:91) |
| 466 | T | 10 | CTTATATAGCTGCGCGGGAAt | | |
| | | | (SEQ ID NO:92) | | |
| 468 | A | 14 | AATCTTACTTATCGAACCGGACTTa | TTTTGCTTGTTGC CC | Fwd. AAATGAGACGGTTTGGGGAGCGAG |
| | | | (SEQ ID NO:93) | (SEQ ID NO:94) | (SEQ ID NO:95) |
| | | | | | Rev. GTGACAGAGAATGAGTTTGCGATG |
| | | | | | (SEQ ID NO:96) |
| 468 | C | 14 | AATCTTACTTATCGAACCGGACTTc | | |
| | | | (SEQ ID NO:97) | | |
| 505 | A | 44 | CATCCTCCAGCGCCCTCa | GTCACAGCACTG | Fwd. ATATTTCACCTGGCCTTTGAG^{e} |
| | | | (SEQ ID NO:98) | (SEQ ID NO:99) | (SEQ ID NO:100) |
| | | | | | Rev. TACAGTCTCATGAGGATAGCCC^{f} |
| | | | | | (SEQ ID NO:101) |
| 505 | G | 44 | ATCCTCCAGCGCCCTCg | | |
| | | | (SEQ ID NO:102) | | |
| 507 | C | 46 | GATCACTTTTCCACAGCTGGAc | CACCTTGAGAATG | Fwd. GCTCTAAAGAGAAGCTCACAGC^{e} |
| | | | (SEQ ID NO:103) | (SEQ ID NO:104) | (SEQ ID NO:105) |
| | | | | | Rev. CACCTGAGATTAAAAGGTCTGC^{f} |
| | | | | | (SEQ ID NO:106) |
| 507 | G | 46 | GATCACTTTTCCACAGCTGGAg | | |
| | | | (SEQ ID NO:107) | | |
| 511 | C | 49 | ATGCAGGAGAATGACCAGCc | GTCCTGCACCTG | Fwd. CTAAAGACAAGTCTCCAGTGGC^{e} |
| | | | (SEQ ID NO:108) | (SEQ ID NO:109) | (SEQ ID NO:110) |
| | | | | | Rev. GTCATGACAGCTACAGGAAAGG^{f} |
| | | | | | (SEQ ID NO:111) |
| 511 | T | 49 | GATGCAGGAGAATGACCAGCt | | |
| | | | (SEQ ID NO:112) | | |

^{a}All sequences are written 5′ to 3′
^{b}Each SNP uses two target oligo
^{c}The polymorphic base at the 3′ end of the sequence is shown in lower case.
^{d}Each reporter sequence has a 5′ PO₄ and 3′ biotin.
^{e}Each of these forward sequence primers has a 5′ sequence: 5′-tgtaaaacgacggccagt-3′.
^{f}Each of these reverse sequence primers has a 5′ sequence: 5′caggaaacagctatgacc-3′.

What is claimed is:

1. A method of detecting a result from an identification reaction to identify a selected nucleotide in a target nucleic acid comprising:
a. contacting a target oligonucleotide comprising a first complementarity region and a second complementarity region, wherein the second complementarity region is 5′ of the first complementarity region and wherein the first complementarity region comprises a region complementary to a section of the target nucleic acid that is directly 3′ of and adjacent to the selected nucleotide, with a sample comprising the target nucleic acid, under hybridization conditions that allow the formation of a first hybridization product;
b. performing, in the presence of a selectively labeled reporter probe, a selected identification reaction with the first hybridization product to determine the identity of the selected nucleotide, wherein a selectively labeled detection product comprising the target oligonucleotide and the reporter probe can be formed;
c. isolating the detection product by contacting the detection product with a capture oligonucleotide that is covalently coupled directly or indirectly to a mobile solid support, wherein the capture oligonucleotide comprises a nucleic acid sequence complementary to the second complementarity region of the target oligonucleotide, under hybridization conditions to form a second hybridization product; and
d. detecting the label of the labeled detection product in the second hybridization product,
the presence of the label indicating the identity of the selected nucleotide in the target nucleic acid.

2. The method of claim 1, wherein the capture oligonucleotide has a GC content of about 50% or greater.

3. The method of claim 1, wherein the capture oligonucleotide has a Tₘ of about 60 to 70° C.

4. The method of claim 1, wherein the capture oligonucleotide comprises a sequence not present in a cell that contains the target nucleic acid.

5. The method of claim 4, wherein the target nucleic acid is a sequence present in mammalian cells and the capture oligonucleotide comprises an oligonucleotide sequence present in a bacterium.

6. The method of claim 4, wherein the capture oligonucleotide comprises an oligonucleotide sequence present in Mycobacterium tuberculosis.

7. The method of claim 1, wherein the capture oligonucleotide further comprises a 5′ amine group.

8. The method of claim 1, wherein the capture oligonucleotide further comprises a luciferase cDNA.

9. The method of claim 1, wherein the second complementary region of the target oligonucleotide comprises a nucleic acid of at least 8 nucleotides.

10. The method of claim 1, wherein the second complementarity region of the target oligonucleotide comprises a nucleic acid having the sequence selected from the group consisting of SEQ ID NO:1-58.

11. The method of claim 1, wherein the identification reaction is a single base chain extension reaction.

12. The method of claim 11, wherein the single base chain extension reaction comprises performing a primer extension reaction with the first hybridization product; wherein the detectably labeled reporter probe comprises an identified, chain-terminating nucleotide under conditions for primer extension; and wherein the presence of a label in the second hybridization product indicates the incorporation of the labeled nucleotide into the first hybridization product, the identity of the incorporated labeled nucleotide indicating the identity of the nucleotide complementary to the selected nucleotide, thus identifying the selected nucleotide in the target nucleic acid.

13. The method of claim 12, wherein the chain-terminating nucleotide is a 3′deoxynucleotide, a 3′deoxyribonucleotide, a thiol nucleotide derivative or a dideoxynucleotide.

14. The method of claim 12, wherein the chain terminating nucleotide is a dideoxynucleotide and the primer extension is performed in the presence of one labeled, identified dideoxynucleotide and three different, non-labeled dideoxynucleotides.

15. The method of claim 12, wherein the chain terminating nucleotide is a dideoxynucleotide and the primer extension is performed in the presence of one labeled, identified dideoxynucleotide and two different, non-labeled dideoxynucleotides.

16. The method of claim 12, wherein the chain terminating nucleotide is a dideoxynucleotide and the primer extension is performed in the presence of one labeled, identified dideoxynucleotide and one different, non-labeled dideoxynucleotides.

17. The method of claim 12, wherein the chain terminating nucleotide is a dideoxynucleotide and the primer extension is performed in the presence of one labeled, identified dideoxynucleotide and in the absence of any different, non-labeled dideoxynucleotides.

18. The method of claim 12, wherein the label of the chain-terminating nucleotide is selected from the group consisting of a hapten, radiolabel, and fluorescent label.

19. The method of claim 1, wherein the identification reaction is an oligonucleotide ligation reaction.

20. The method of claim 19, wherein the oligonucleotide ligation reaction comprises performing a ligation reaction between the target oligonucleotide and the reporter probe; wherein the selectively labeled reporter probe comprises a sequence that is complementary to a section of the target nucleic acid directly 5′ the selected nucleotide and that terminates at its 3′ end in an identified test nucleotide positioned to base-pair with the selected nucleotide of the target nucleic acid, under conditions for ligation; and wherein the detection comprises detecting the presence or absence of a label incorporated into the second hybridization product, the presence of a label indicating the incorporation of the labeled reporter probe in the reaction product, and the identity of the incorporated labeled reporter probe indicating the identity of the nucleotide complementary to the selected nucleotide, thus identifying the selected nucleotide in the target nucleic acid.

21. The method of claim 20, wherein the reporter probe comprises one or more nucleotides and has a 5′ phosphate group.

22. The method of claim 21, wherein the reporter probe further comprises a 3′ label.

23. The method of claim 20, wherein the reporter probe is an oligonucleotide.

24. The method of claim 23, wherein the oligonucleotide is an 8-mer.

25. The method of claim 1, wherein the identification reaction is an allelle-specific polymerization reaction.

26. The method of claim 25, wherein the allelle-specific polymerization reaction comprises performing a polymerization reaction with a non-proof reading polymerase, wherein a primer for the reaction comprises the first complementarity region of the target oligonucleotide, wherein the reporter probe comprises one or more selectively labeled deoxynucleotides, and wherein the detection comprises detecting the presence or absence of a label incorporated into the second hybridization product, the presence of the label indicating the extension of the primer and the identity of the label indicating the nucleotide complementary to the selected nucleotide, thus identifying the selected nucleotide in the target nucleic acid.

27. The method of claim 1, wherein the target nucleic acid is an oligonucleotide, a 16s ribosomal RNA, a PCR product, a DNA fragment, an RNA molecule, a cDNA molecule or a cRNA molecule, the nucleic acid primer is an oligonucleotide, a PCR product, a DNA fragment, an RNA molecule, a cDNA molecule, a cRNA molecule, or genomic DNA.

28. The method of claim 1, wherein the mobile solid support is a bead.

29. The method of claim 1, further comprising performing the selected identification reaction in the presence of more than one reporter probe, wherein each reporter probe comprises a different detectable label and a different nucleotide complementary to the selected nucleotide of the target nucleic acid, to produce detection products with different labels, and detecting the different labels of the labeled detection products in the second hybridization products, the presence of each label indicating the identity of each selected nucleotide in the target nucleic acid.

30. The method of claim 29, further comprising quantifying the different labels of the labeled detection products in the second hybridization products, the quantity of the different labels indicating the relative occurrence of each selected nucleotide in the target nucleic acid.

31. The method of claim 29, wherein more than one capture oligonucleotide is covalently coupled to the mobile solid support and wherein each second hybridization product can comprise one or more labels.

32. The method of claim 31, further comprising quantifying the different labels of the labeled detection products in the second hybridization products, the quantity of the different labels indicating the relative occurrence of each selected nucleotide in the target nucleic acid.

33. A method of detecting a result from an identification reaction to identify one or more selected nucleotides in one or more target nucleic acids comprising:
a. contacting one or more specific target oligonucleotides, wherein each target oligonucleotide comprises a first specific complementarity region and a second specific complementarity region, wherein the second complementarity region of each target oligonucleotide is 5′ of the first complementarity region and wherein the first complementarity region of each target oligonucleotide comprises a sequence that is complementary to a section of the target nucleic acid directly 3′ of the selected nucleotide and that terminates at its 3′ end in an identified test nucleotide positioned to base-pair with the selected nucleotide of the target nucleic acid, with a sample comprising one or more target nucleic acids, under hybridization conditions, to form first hybridization products;
b. performing, in the presence of one or more selectively labeled reporter probes, a selected identification reaction with the first hybridization products, wherein selectively labeled detection products comprising the first complementarity region of the target oligonucleotides and the reporter probes can be formed;
c. isolating the detection products by contacting the detection products, under hybridization conditions to form second hybridization products, with specific capture oligonucleotides that are covalently coupled directly or indirectly to specific detectably tagged mobile solid supports, wherein each capture oligonucleotide comprises a nucleic acid sequence complementary to a second complementarity region of a specific target oligonucleotide and wherein the detectable tag is specific for each capture oligonucleotide; and
d. detecting the labels of the labeled detection product in the second hybridization product and the detectable tags of the mobile solid support in the same second hybridization product,
the presence of the label and the specific detectable tag in the same second hybridization product indicating the identity of the selected nucleotides in the target nucleic acid.

34. The method of claim 33, wherein each capture oligonucleotide has a GC content of about 50% or greater.

35. The method of claim 33, wherein each capture oligonucleotide has a Tₘ of about 60 to 70° C.

36. The method of claim 33, wherein each capture oligonucleotide comprises a sequence not present in a cell that contains the target nucleic acid.

37. The method of claim 36, wherein the target nucleic acid is a sequence present in mammalian cells and the capture oligonucleotide comprises an oligonucleotide sequence present in a bacterium.

38. The method of claim 37, wherein each capture oligonucleotide comprises an oligonucleotide sequence present in Mycobacterium tuberculosis.

39. The method of claim 33, wherein each capture oligonucleotide further comprises a 5′ amine group.

40. The method of claim 33, wherein each capture oligonucleotide further comprises a luciferase cDNA.

41. The method of claim 33, wherein the second complementarity region of each target oligonucleotide comprises a nucleic acid of at least 8 nucleotides.

42. The method of claim 33, wherein the second complementarity region of each target oligonucleotide comprises a nucleic acid having the sequence selected from the group consisting of SEQ ID NO:1-58.

43. The method of claim 33, wherein the identification reaction is a single base chain extension reaction.

44. The method of claim 43, wherein the single base chain extension reaction comprises performing a primer extension reaction with the first hybridization products; wherein each detectably labeled reporter probe comprises an identified, chain-terminating nucleotide under conditions for primer extension; and wherein the presence of a selected label in the second hybridization product indicates the incorporation of the labeled nucleotide into the first hybridization product, the identity of the incorporated labeled nucleotide indicating the identity of the nucleotide complementary to the selected nucleotide, thus identifying the selected nucleotide in the target nucleic acid.

45. The method of claim 44, wherein each chain-terminating nucleotide is a 3′deoxynucleotide, a 3′deoxyribonucleotide, a thiol nucleotide derivative or a dideoxynucleotide.

46. The method of claim 45, wherein each chain terminating nucleotide is a dideoxynucleotide and the primer extension is performed in the presence of one labeled, identified dideoxynucleotide and three different, non-labeled dideoxynucleotides.

47. The method of claim 45, wherein each chain terminating nucleotide is a dideoxynucleotide and the primer extension is performed in the presence of one labeled, identified dideoxynucleotide and two different, non-labeled dideoxynucleotides.

48. The method of claim 45, wherein each chain terminating nucleotide is a dideoxynucleotide and the primer extension is performed in the presence of one labeled, identified dideoxynucleotide and one different, non-labeled dideoxynucleotides.

49. The method of claim 45, wherein each chain terminating nucleotide is a dideoxynucleotide and the primer extension is performed in the presence of one labeled, identified dideoxynucleotide and in the absence of any different, non-labeled dideoxynucleotides.

50. The method of claim 45, wherein the label of each chain-terminating nucleotide is selected from the group consisting of a hapten, radiolabel, and fluorescent label.

51. The method of claim 33, wherein the identification reaction is an oligonucleotide ligation reaction.

52. The method of claim 51, wherein the oligonucleotide ligation reaction comprises performing a ligation reaction between the target oligonucleotides and the reporter probes.

53. The method of claim 52, wherein the reporter probe comprises one or more nucleotides and has a 5′ phosphate group.

54. The method of claim 53, wherein the reporter probe further comprises a 3′ label.

55. The method of claim 52, wherein the reporter probe is an oligonucleotide.

56. The method of claim 55, wherein the oligonucleotide is an 8-mer.

57. The method of claim 33, wherein the identification reaction is an allelle-specific polymerization reaction.

58. The method of claim 57, wherein the allelle-specific polymerization reaction comprises performing a polymerization reaction with a non-proof reading polymerase, wherein each primer for the reaction comprises the first complementarity region of the target oligonucleotide, wherein the reporter probe comprises one or more selectively labeled deoxynucleotides, and wherein the detection comprises detecting the presence or absence of a label incorporated into the second hybridization product, the presence of the label indicating the extension of the primer and the identity of the label indicating the nucleotide complementary to the selected nucleotide, thus identifying the selected nucleotide in the target nucleic acid.

59. The method of claim 33, wherein the target nucleic acid is an oligonucleotide, a 16s ribosomal RNA, a PCR product, a DNA fragment, an RNA molecule, a cDNA molecule or a cRNA molecule, the nucleic acid primer is an oligonucleotide, a PCR product, a DNA fragment, an RNA molecule, a cDNA molecule, a cRNA molecule, or genomic DNA.

60. The method of claim 33, wherein the mobile solid support is a bead.

61. The method of claim 83, further comprising quantifying the labels and specific detectable tags in the second hybridization products, the quantity of the labels and specific detectable tags in the second hybridization products indicating the relative occurrence of each selected nucleotide in the target nucleic acid.

62. A method of determining one or more selected nucleotide polymorphisms in genomic DNA comprising:
a′. performing an amplification of the genomic DNA using a first nucleic acid primer comprising a region complementary to a section of one strand of the nucleic acid that is 5′ of the selected nucleotide, and a second nucleic acid primer complimentary to a section of the opposite strand of the nucleic acid downstream of the selected nucleotide, under conditions for specific amplification of the region of the selected nucleotide between the two primers, to form a PCR product;
a″ performing an amplification of the genomic DNA using as a primer an oligonucleotide comprising a first region having a T7 RNA polymerase promoter and a second region complementary to a section of one strand of the nucleic acid that is directly 5′ of the selected nucleotide, and using T7 RNA polymerase to amplify one strand into cRNA and using reverse transcriptase to amplify the second strand complementary to the cRNA strand, under conditions for specific amplification of the region of the nucleotide between the two primers, to form a cRNA amplification product; or
a′″. treating genomic DNA to decrease viscosity; and
b. contacting a sample comprising one or more PCR products, one or more cRNA amplification products, or treated genomic DNA with one or more specific target oligonucleotides, wherein each target oligonucleotide comprises a first specific complementarity region and a second specific complementarity region, wherein the second complementarity region of each target oligonucleotide is 5′ of the first complementarity region, and wherein the first complementarity region of each target oligonucleotide comprises a sequence that is complementary to a section of the target nucleic acid directly 5′ of the selected nucleotide and that terminates at its 3′ end in an identified test nucleotide positioned to base-pair with a selected nucleotide of the PCR products, cRNA amplification products, or treated genomic DNA, under hybridization conditions, to form first hybridization products;
c. performing, in the presence of one or more selectively labeled reporter probes, a selected identification reaction with the first hybridization products, wherein selectively labeled detection products comprising the first complementarity region of the target oligonucleotides and the reporter probes can be formed;
b. isolating the detection products by contacting the detection products, under hybridization conditions to form a second hybridization product, with specific oligonucleotides that are covalently coupled directly or indirectly to specific detectably tagged mobile solid supports, wherein each capture oligonucleotide comprises a nucleic acid sequence complementary to a second complementarity region of a specific target oligonucleotide and wherein the detectable tag is specific for each capture oligonucleotide; and
c. detecting the label of the labeled detection product in the second hybridization product and the detectable tag of the mobile solid support in the same second hybridization product, the presence of the label and the specific detectable tag in the same second hybridization product indicating the identity of the selected nucleotide in the specific PCR products, cRNA amplification products, or treated genomic DNA; and
d. comparing the identities of the identified nucleotides with a non-polymorphic nucleotide, a different identity of the identified nucleotide from that of the non-polymorphic nucleotide indicating one or more polymorphisms in the genomic DNA.

63. The method of claim 62, wherein each capture oligonucleotide has a GC content of about 50% or greater.

64. The method of claim 62, wherein each capture oligonucleotide has a Tₘ of about 60 to 70° C.

65. The method of claim 62, wherein each capture oligonucleotide comprises a sequence not present in a cell that contains the target nucleic acid.

66. The method of claim 65, wherein the target nucleic acid is a sequence present in mammalian cells and the capture oligonucleotide comprises an oligonucleotide sequence present in a bacterium.

67. The method of claim 66, wherein each capture oligonucleotide comprises an oligonucleotide sequence present in Mycobacterium tuberculosis.

68. The method of claim 62, wherein each capture oligonucleotide further comprises a 5′ amine group.

69. The method of claim 62, wherein each capture oligonucleotide further comprises a luciferase cDNA.

70. The method of claim 62, wherein the second complementarity region of each target oligonucleotide comprises a nucleic acid of at least 8 nucleotides.

71. The method of claim 62, wherein the second complementarity region of each target oligonucleotide comprises a nucleic acid having the sequence selected from the group consisting of SEQ ID NO:1-58.

72. The method of claim 62, wherein the identification reaction is a single base chain extension reaction.

73. The method of claim 72, wherein the single base chain extension reaction comprises performing a primer extension reaction with the first hybridization products; wherein each detectably labeled reporter probe comprises an identified, chain-terminating nucleotide under conditions for primer extension.

74. The method of claim 73, wherein each chain-terminating nucleotide is a 3′deoxynucleotide, a 3′deoxyribonucleotide, a thiol nucleotide derivative or a dideoxynucleotide.

75. The method of claim 74, wherein each chain terminating nucleotide is a dideoxynucleotide and the primer extension is performed in the presence of one labeled, identified dideoxynucleotide and three different, non-labeled dideoxynucleotides.

76. The method of claim 74, wherein each chain terminating nucleotide is a dideoxynucleotide and the primer extension is performed in the presence of one labeled, identified dideoxynucleotide and two different, non-labeled dideoxynucleotides.

77. The method of claim 74, wherein each chain terminating nucleotide is a dideoxynucleotide and the primer extension is performed in the presence of one labeled, identified dideoxynucleotide and one different, non-labeled dideoxynucleotides.

78. The method of claim 74, wherein each chain terminating nucleotide is a dideoxynucleotide and the primer extension is performed in the presence of one labeled, identified dideoxynucleotide and in the absence of any different, non-labeled dideoxynucleotides.

79. The method of claim 74, wherein the label of each chain-terminating nucleotide is selected from the group consisting of a hapten, radiolabel, and fluorescent label.

80. The method of claim 62, wherein the identification reaction is an oligonucleotide ligation reaction.

81. The method of claim 80, wherein the oligonucleotide ligation reaction comprises performing a ligation reaction between the target oligonucleotides and the reporter probes.

82. The method of claim 81, wherein the reporter probe comprises one or more nucleotides and has a 5′ phosphate group.

83. The method of claim 82, wherein the reporter probe further comprises a 3′ label.

84. The method of claim 81, wherein the reporter probe is an oligonucleotide.

85. The method of claim 84, wherein the oligonucleotide is an 8-mer.

86. The method of claim 62, wherein the identification reaction is an allelle-specific polymerization reaction.

87. The method of claim 86, wherein the allelle-specific polymerization reaction comprises performing a allelle-specific polymerization reaction with a non-proof reading polymerase, wherein each primer for the allelle-specific polymerization reaction comprises the first complementarity region of the target oligonucleotide, wherein the reporter probe comprises one or more selectively labeled deoxynucleotides, and wherein the detection comprises detecting the presence or absence of a label incorporated into the second hybridization product, the presence of the label indicating the extension of the primer and the identity of the label indicating the nucleotide complementary to the selected nucleotide.

88. The method of claim 62, wherein the target nucleic acid is an oligonucleotide, a 16s ribosomal RNA, a PCR product, a DNA fragment, an RNA molecule, a cDNA molecule or a cRNA molecule, the nucleic acid primer is an oligonucleotide, a PCR product, a DNA fragment, an RNA molecule, a cDNA molecule, a cRNA molecule, or genomic DNA.

89. The method of claim 62, wherein the mobile solid support is a bead.

90. The method of claim 62, wherein more than one capture oligonucleotide is covalently coupled to the mobile solid support and wherein each second hybridization product can comprise one or more labels.

91. The method of claim 62, further comprising quantifying the labels and specific tags in the second hybridization products, the quantity of the labels and specific detectable tags in the same second hybridization products indicating the relative occurrence of each selected nucleotide in the target nucleic acid.

92. A method of detecting results from a cleavase/signal release reaction to identify one or more selected nucleotides in a target nucleic acid comprising:
a. contacting a sample comprising the target nucleic acid with (i) one or more signal probes, wherein each signal probe comprises a first complementarity region and a selected second complementarity region that is specific for a test nucleotide, wherein the second complementarity region is 5′ of the first complementarity region and comprises a donor fluorophore, and wherein the first complementarity region comprises (a) a sequence that is complementary to a section of the target nucleic acid that is directly 5′ of the selected nucleotide, (b) the test nucleotide at its 5′ end that is positioned to base-pair with the selected nucleotide of the target nucleic acid, and (c) a quenching fluorophore that is located 3′ to the identified test nucleotide and (ii) more than one invader oligonucleotide, wherein each invader oligonucleotide comprises (a) a sequence that is complementary to a section of the target nucleic acid that is directly 3′ of the selected nucleotide and (b) the identified test nucleotide at its 5′ end that is positioned to base-pair with the selected nucleotide of the target nucleic acid, under hybridization conditions that allow the formation of overlapping hybridization products between the first complementarity region of the signal probes and the section of the target nucleic acid complementary to the first complementarity region of the signal probes and between the invader oligonucleotides and the complementary section of the target nucleic acid, to form the overlapping hybridization products, wherein the overlapping hybridization products overlap at the selected nucleotide;
b. performing specific cleavage reactions comprising contacting the overlapping hybridization products with a nuclease that specifically cleaves the overlapping hybridization products formed when the identified test nucleotide and selected nucleotide are complementary, and releasing detection products comprising the specific second complementary regions and the identified test nucleotide of the first complementarity region of the signal probes;
c. isolating the detection products by contacting the detection products, under hybridization conditions to form non-overlapping second hybridization products, with specific capture oligonucleotides that are covalently coupled directly or indirectly to specific detectably tagged mobile solid supports, wherein each capture oligonucleotide comprises a nucleic acid sequence complementary to a specific second complementarity region of a specific signal probe and wherein the detectable tag is specific for each capture oligonucleotide; and
d. detecting the presence of the donor fluorophore and the absence of the quenching fluorophore and the presence of the detectable tags of the mobile solid support in the same in the non-overlapping hybridization products,
the presence of the specific detectable tag and the donor fluorophore and the absence of the quenching fluorophore indicating the identity of the selected nucleotide in the target nucleic acid.

93. The method of claim 92, further comprising repetitions of steps (a) and (b) above to increase the amount of detection product.

94. The method of claim 92, wherein each capture oligonucleotide has a GC content of about 50% or greater.

95. The method of claim 92, wherein each capture oligonucleotide has a Tₘ of about 60 to 70° C.

96. The method of claim 92, wherein each capture oligonucleotide comprises a sequence not present in a cell that contains the target nucleic acid.

97. The method of claim 96, wherein the target nucleic acid is a sequence present in mammalian cells and the capture oligonucleotide comprises an oligonucleotide sequence present in a bacterium.

98. The method of claim 97, wherein each capture oligonucleotide comprises an oligonucleotide sequence present in Mycobacterium tuberculosis.

99. The method of claim 98, wherein each capture oligonucleotide further comprises a 5′ amine group.

100. The method of claim 98, wherein each capture oligonucleotide further comprises a luciferase cDNA.

101. The method of claim 92, wherein each capture oligonucleotide is coupled at either its 5′ or 3′ end to the mobile solid support.

102. The method of claim 92, wherein the second complementarity region of each signal probe comprises a nucleic acid of at least 8 nucleotides.

103. The method of claim 92, wherein the second complementarity region of each signal probe comprises a nucleic acid having the sequence selected from the group consisting of SEQ ID NO:1-58.

104. The method of claim 92, further comprising quantifying the occurrence of specific detectable tags and donor fluorophores and the absence of quenching fluorophores in the same non-overlapping hybridization products indicating the relative occurrence of each selected nucleotide in the target nucleic acid.

105. A method of detecting results from a polymerase/repair reaction to identify selected nucleotides in a target nucleic acid comprising:
a. contacting a sample comprising the target nucleic acid with (i) one or more signal probes, wherein each signal probe comprises a first complementarity region and a selected second complementarity region that is specific for a test nucleotide, wherein the second complementarity region is 3′ of the first complementarity region, and wherein the first complementarity region comprises (a) a sequence that is complementary to a section of the target nucleic acid that is directly 5′ of the selected nucleotide, (b) the identified test nucleotide at the 5′ end of the signal probe, wherein the test nucleotide is positioned to base-pair with the selected nucleotide of the target nucleic acid, (c) a thiol site located 3′ of the test nucleotide, (d) a donor fluorophore that is located 3′ to the thiol site, (d) a quenching fluorophore that is located 5′ to the thiol site and 3′ to the test nucleotide, under hybridization conditions that allow the formation of first hybridization products between the first complementarity region of the signal probes and the section of the target nucleic acid complementary to the first complementarity region of the signal probes;
b. performing a polymerase/repair reaction comprising contacting the first hybridization products with a Taq polymerase that cleaves the signal probes at the thiol site when the test nucleotide and the selected nucleotide are complementary and releases detection products comprising the second complementary region and the portion of the first complementary region of the signal probes that contain the donor fluorophore but lack the quenching fluorophore;
c. isolating the detection products by contacting the detection products, under hybridization conditions to form second hybridization products, with specific capture oligonucleotides that are covalently coupled directly or indirectly to specific detectably tagged mobile solid supports, wherein each capture oligonucleotide comprises a nucleic acid sequence complementary to a specific second complementarity region of a specific signal probe and wherein the detectable tag is specific for each capture oligonucleotide; and
d. detecting the presence of the donor fluorophore, the absence of the quenching fluorophore, and the presence of the specific detectable tags of the mobile solid support in the same second hybridization products,
the presence of the specific detectable tag and the donor fluorophore and the absence of the quenching fluorophore indicating the identity of the selected nucleotides in the target nucleic acid.

106. The method of claim 105, further comprising repetitions of steps (a) and (b) above to increase the amount of detection product.

107. The method of claim 105, wherein each capture oligonucleotide has a GC content of about 50% or greater.

108. The method of claim 105, wherein each capture oligonucleotide has a Tₘ of about 60 to 70° C.

109. The method of claim 105, wherein each capture oligonucleotide comprises a sequence not present in a cell that contains the target nucleic acid.

110. The method of claim 105, wherein the target nucleic acid is a sequence present in mammalian cells and the capture oligonucleotide comprises an oligonucleotide sequence present in a bacterium.

111. The method of claim 110, wherein each capture oligonucleotide comprises an oligonucleotide sequence present in Mycobacterium tuberculosis.

112. The method of claim 110, wherein each capture oligonucleotide further comprises a 5′ amine group.

113. The method of claim 110, wherein each capture oligonucleotide further comprises a luciferase cDNA.

114. The method of claim 105, wherein the second complementarity region of each signal probe comprises a nucleic acid of at least 8 nucleotides.

115. The method of claim 105, wherein the second complementarity region of each signal probe comprises a nucleic acid having the sequence selected from the group consisting of SEQ ID NO:1-58.

116. The method of claim 105, further comprising quantifying the occurrence of specific detectable tags and donor fluorophores and the absence of quenching fluorophores in the same non-overlapping hybridization products indicating the relative occurence of each selected nucleotide in the target nucleic acid.

117. A method of detecting selected microbial contaminants in a sample comprising:
a. contacting the sample with one or more target oligonucleotides, wherein each target oligonucleotide comprises a first complementarity region and a second complementarity region, wherein the first complementarity region comprises a region complementary to a section of a nucleic acid that is specific to a selected microbial contaminant and wherein the second complementarity region comprises a region complementary to a specific labeled reporter probe, under hybridization conditions that allow the formation of hybridization products between the first complementarity region of the target oligonucleotides and a region of the microbial nucleic acid complementary to the first complementarity region of the target oligonucleotide, to form first hybridization products;
b. performing, in the presence of one or more labeled reporter probes, a selected identification reaction with the first hybridization products, wherein selectively labeled detection products can be formed and wherein each detection product comprises the second complementary of a specific target oligonucleotide and a label;
c. isolating the detection products by contacting the detection products with specific capture oligonucleotides that are covalently coupled directly or indirectly to specific detectably tagged mobile solid supports, wherein each capture oligonucleotide comprises a nucleic acid sequence complementary to a second complementarity region of a specific target oligonucleotide and wherein the detectable tag is specific for each capture oligonucleotide; and
d. detecting the labels of the labeled detection product in the second hybridization product and the detectable tags of the mobile solid support in the same second hybridization product,
the presence of the label and the specific detectable tag in the same second hybridization product indicating the identity of microbial contaminants in the sample.

118. The method of claim 117, wherein one of the selected microbial contaminants is S. aureus.

119. The method of claim 118, wherein the first complementarity region complementary to a section of a nucleic acid that is specific to S. aureus has the nucleic acid sequence of SEQ ID NO:60.

120. The method of claim 118, wherein the first complementarity region complementary to a section of a nucleic acid that is specific to S. aureus has the nucleic acid sequence of SEQ ID NO:61.

121. The method of claim 117, wherein one of the selected microbial contaminants is B. cepacia.

122. The method of claim 121, wherein the first complementarity region complementary to a section of a nucleic acid that is specific to B. cepacia has the nucleic acid sequence of SEQ ID NO:62.

123. The method of claim 121, wherein the first complementarity region complementary to a section of a nucleic acid that is specific to B. cepacia has the nucleic acid sequence of SEQ ID NO:63.

124. The method of claim 117, wherein one of the selected microbial contaminants is either E. coli or Pseudomonas.

125. The method of claim 124, wherein the first complementarity region complementary to a section of a nucleic acid that is specific to E. coli or Pseudomonas has the nucleic acid sequence of SEQ ID NO:64.

126. The method of claim 124, wherein the first complementarity region complementary to a section of a nucleic acid that is specific to E. coli or Pseudomonas has the nucleic acid sequence of SEQ ID NO:65.

127. The method of claim 117, wherein one of the selected microbial contaminants is either or Pseudomonas or B. cepacia.

128. The method of claim 127, wherein the first complementarity region complementary to a section of a nucleic acid that is specific to Pseudomonas or B. cepacia has the nucleic acid sequence of SEQ ID NO:66.

129. The method of claim 127, wherein the first complementarity region complementary to a section of a nucleic acid that is specific to Pseudomonas or B. cepacia has the nucleic acid sequence of SEQ ID NO:67.


SEQUENCE LISTING (112 sequences)

SEQ ID NO: 1 - DNA, 25 bp, Artificial Sequence
  Description of Artificial Sequence/Note = synthetic construct
gatgatcgacgagacactctcgcca

SEQ ID NO: 2 - DNA, 25 bp, Artificial Sequence
  Description of Artificial Sequence/Note = synthetic construct
cggtcgacgagctgccgcgcaagat

SEQ ID NO: 3 - DNA, 25 bp, Artificial Sequence
  Description of Artificial Sequence/Note = synthetic construct
gacattcgcgatcgccgcccgcttt

SEQ ID NO: 4 - DNA, 25 bp, Artificial Sequence
  Description of Artificial Sequence/Note = synthetic construct
cggtatcgcgaccgcatcccaatct

SEQ ID NO: 5 - DNA, 25 bp, Artificial Sequence
  Description of Artificial Sequence/Note = synthetic construct
gctcgaagaggcgctacagatcctc

SEQ ID NO: 6 - DNA, 25 bp, Artificial Sequence
  Description of Artificial Sequence/Note = synthetic construct
caccgccagctcggcttcgagttcg

SEQ ID NO: 7 - DNA, 25 bp, Artificial Sequence
  Description of Artificial Sequence/Note = synthetic construct
cgactccctgtttgtgatggaccac

SEQ ID NO: 8 - DNA, 25 bp, Artificial Sequence
  Description of Artificial Sequence/Note = synthetic construct
cttttcccgtccgtcatcgctcaag

SEQ ID NO: 9 - DNA, 25 bp, Artificial Sequence
  Description of Artificial Sequence/Note = synthetic construct
ggctgggtctacagatccccaactt

SEQ ID NO: 10 - DNA, 25 bp, Artificial Sequence
  Description of Artificial Sequence/Note = synthetic construct
gaacctttcgcttcaccggccgatc

SEQ ID NO: 11 - DNA, 25 bp, Artificial Sequence
  Description of Artificial Sequence/Note = synthetic construct
tttcggcacgcgcgggatcaccatc

SEQ ID NO: 12 - DNA, 25 bp, Artificial Sequence
  Description of Artificial Sequence/Note = synthetic construct
ctcggtggtgctgacggtgcaatcc

SEQ ID NO: 13 - DNA, 25 bp, Artificial Sequence
  Description of Artificial Sequence/Note = synthetic construct
tcaacgtgccagcgccgtcctggga

SEQ ID NO: 14 - DNA, 25 bp, Artificial Sequence
  Description of Artificial Sequence/Note = synthetic construct
gcgaaggaactcgacgtggacgccg

SEQ ID NO: 15 - DNA, 25 bp, Artificial Sequence
  Description of Artificial Sequence/Note = synthetic construct
cggggataccgatctcgggcgcaca

SEQ ID NO: 16 - DNA, 25 bp, Artificial Sequence
  Description of Artificial Sequence/Note = synthetic construct
ggagcttacgccatcacgatgcgat

SEQ ID NO: 17 - DNA, 25 bp, Artificial Sequence
  Description of Artificial Sequence/Note = synthetic construct
cgtggcggtgcggagtttccccgaa

SEQ ID NO: 18 - DNA, 25 bp, Artificial Sequence
  Description of Artificial Sequence/Note = synthetic construct
cgatccaacgcactggccaaaccta

SEQ ID NO: 19 - DNA, 25 bp, Artificial Sequence
  Description of Artificial Sequence/Note = synthetic construct
ctgaatcctccaaccgggttgtcga

SEQ ID NO: 20 - DNA, 25 bp, Artificial Sequence
  Description of Artificial Sequence/Note = synthetic construct
ttcggcgctggcgtaaagcttttgg

SEQ ID NO: 21 - DNA, 25 bp, Artificial Sequence
  Description of Artificial Sequence/Note = synthetic construct
gtaaatctccagcggaagggtacgg

SEQ ID NO: 22 - DNA, 25 bp, Artificial Sequence
  Description of Artificial Sequence/Note = synthetic construct
ccggctttgaactgctcaccgatct

SEQ ID NO: 23 - DNA, 25 bp, Artificial Sequence
  Description of Artificial Sequence/Note = synthetic construct
actacgcaacaccgaacggataccc

SEQ ID NO: 24 - DNA, 25 bp, Artificial Sequence
  Description of Artificial Sequence/Note = synthetic construct
ggaccaatggtcccattgaccaggt

SEQ ID NO: 25 - DNA, 25 bp, Artificial Sequence
  Description of Artificial Sequence/Note = synthetic construct
caacgctgagcgcgtcactgacata

SEQ ID NO: 26 - DNA, 25 bp, Artificial Sequence
  Description of Artificial Sequence/Note = synthetic construct
gagacaaaggtctgcgccagcacca

SEQ ID NO: 27 - DNA, 25 bp, Artificial Sequence
  Description of Artificial Sequence/Note = synthetic construct
tggccacactgtccatttgcgcggt

SEQ ID NO: 28 - DNA, 25 bp, Artificial Sequence
  Description of Artificial Sequence/Note = synthetic construct
ccttgcgacgtgtcaagttggggtc

SEQ ID NO: 29 - DNA, 25 bp, Artificial Sequence
  Description of Artificial Sequence/Note = synthetic construct
aggttagggtcgcgccaaactctcc

SEQ ID NO: 30 - DNA, 25 bp, Artificial Sequence
  Description of Artificial Sequence/Note = synthetic construct
acgactgcgaggtgcggtaagcaca

SEQ ID NO: 31 - DNA, 25 bp, Artificial Sequence
  Description of Artificial Sequence/Note = synthetic construct
gcgatcgccgggagatatacccaac

SEQ ID NO: 32 - DNA, 25 bp, Artificial Sequence
  Description of Artificial Sequence/Note = synthetic construct
tcgtgccggactcgagcaccaatac

SEQ ID NO: 33 - DNA, 25 bp, Artificial Sequence
  Description of Artificial Sequence/Note = synthetic construct
gctttagcaccgcgatggcgtagac

SEQ ID NO: 34 - DNA, 25 bp, Artificial Sequence
  Description of Artificial Sequence/Note = synthetic construct
cagccgcggtactgaatgcgatgct

SEQ ID NO: 35 - DNA, 25 bp, Artificial Sequence
  Description of Artificial Sequence/Note = synthetic construct
ccccggatagctgacgaggcttacg

SEQ ID NO: 36 - DNA, 25 bp, Artificial Sequence
  Description of Artificial Sequence/Note = synthetic construct
tccggacaggttggggtgcgtttgg

SEQ ID NO: 37 - DNA, 25 bp, Artificial Sequence
  Description of Artificial Sequence/Note = synthetic construct
cgtagagcaacgcgatacccccgac

SEQ ID NO: 38 - DNA, 25 bp, Artificial Sequence
  Description of Artificial Sequence/Note = synthetic construct
agcagcagtgacaatgccaccgccg

SEQ ID NO: 39 - DNA, 25 bp, Artificial Sequence
  Description of Artificial Sequence/Note = synthetic construct
tcgcccgcggacaccgagaattcga

SEQ ID NO: 40 - DNA, 25 bp, Artificial Sequence
  Description of Artificial Sequence/Note = synthetic construct
gaggcagatccgtaggcgggtgcat

SEQ ID NO: 41 - DNA, 25 bp, Artificial Sequence
  Description of Artificial Sequence/Note = synthetic construct
gcgatagccagtgccgccaatcgtc

SEQ ID NO: 42 - DNA, 25 bp, Artificial Sequence
  Description of Artificial Sequence/Note = synthetic construct
agcggtcaccatggccacgaactgc

SEQ ID NO: 43 - DNA, 25 bp, Artificial Sequence
  Description of Artificial Sequence/Note = synthetic construct
ttgcaacagcagcccgactcgacgg

SEQ ID NO: 44 - DNA, 25 bp, Artificial Sequence
  Description of Artificial Sequence/Note = synthetic construct
tgactccggcgatacgggctccgaa

SEQ ID NO: 45 - DNA, 25 bp, Artificial Sequence
  Description of Artificial Sequence/Note = synthetic construct
accggctacctggtatcggtcccga

SEQ ID NO: 46 - DNA, 25 bp, Artificial Sequence
  Description of Artificial Sequence/Note = synthetic construct
gagcgagcgggcaaacgccagtact

SEQ ID NO: 47 - DNA, 25 bp, Artificial Sequence
  Description of Artificial Sequence/Note = synthetic construct
agtcgaagtgggcggcgtcagactc

SEQ ID NO: 48 - DNA, 25 bp, Artificial Sequence
  Description of Artificial Sequence/Note = synthetic construct
caccaccagtgccgctaccacaacg

SEQ ID NO: 49 - DNA, 25 bp, Artificial Sequence
  Description of Artificial Sequence/Note = synthetic construct
ccgtgttaacggcgcgacgcaagga

SEQ ID NO: 50 - DNA, 25 bp, Artificial Sequence
  Description of Artificial Sequence/Note = synthetic construct
gagtgaacgcagactgcagcgaggc

SEQ ID NO: 51 - DNA, 25 bp, Artificial Sequence
  Description of Artificial Sequence/Note = synthetic construct
cggcggtcttcacgctcaacagcag

SEQ ID NO: 52 - DNA, 25 bp, Artificial Sequence
  Description of Artificial Sequence/Note = synthetic construct
gttgggcccgagcactgcaagcacc

SEQ ID NO: 53 - DNA, 25 bp, Artificial Sequence
  Description of Artificial Sequence/Note = synthetic construct
tcggcgtacgagcacccacacccag

SEQ ID NO: 54 - DNA, 25 bp, Artificial Sequence
  Description of Artificial Sequence/Note = synthetic construct
ccccaaacgtaccaagcccgcgtcg

SEQ ID NO: 55 - DNA, 25 bp, Artificial Sequence
  Description of Artificial Sequence/Note = synthetic construct
atggcaccgacggctggcagaccac

SEQ ID NO: 56 - DNA, 25 bp, Artificial Sequence
  Description of Artificial Sequence/Note = synthetic construct
agccgcgaacaccacgatcgaccgg

SEQ ID NO: 57 - DNA, 25 bp, Artificial Sequence
  Description of Artificial Sequence/Note = synthetic construct
cgcgcgcagctgcagcttgctcatg

SEQ ID NO: 58 - DNA, 25 bp, Artificial Sequence
  Description of Artificial Sequence/Note = synthetic construct
taccggcggcagcaccagcggtaac

SEQ ID NO: 59 - DNA, 20 bp, Artificial Sequence
  Description of Artificial Sequence/Note = synthetic construct
caggccaagtaacttcttcg

SEQ ID NO: 60 - DNA, 21 bp, Artificial Sequence
  Description of Artificial Sequence/Note = synthetic construct
gccggtggagtaaccttttag

SEQ ID NO: 61 - DNA, 22 bp, Artificial Sequence
  Description of Artificial Sequence/Note = synthetic construct
gccggtggagtaaccttttagg

SEQ ID NO: 62 - DNA, 18 bp, Artificial Sequence
  Description of Artificial Sequence/Note = synthetic construct
ctgagaggcgggagtgct

SEQ ID NO: 63 - DNA, 19 bp, Artificial Sequence
  Description of Artificial Sequence/Note = synthetic construct
ctgagaggcgggagtgctc

SEQ ID NO: 64 - DNA, 11 bp, Artificial Sequence
  Description of Artificial Sequence/Note = synthetic construct
aataccgcata

SEQ ID NO: 65 - DNA, 12 bp, Artificial Sequence
  Description of Artificial Sequence/Note = synthetic construct
aataccgcatan

SEQ ID NO: 66 - DNA, 13 bp, Artificial Sequence
  Description of Artificial Sequence/Note = synthetic construct
aataccgcatacg

SEQ ID NO: 67 - DNA, 14 bp, Artificial Sequence
  Description of Artificial Sequence/Note = synthetic construct
aataccgcatacgn

SEQ ID NO: 68 - DNA, 23 bp, Artificial Sequence
  Description of Artificial Sequence/Note = synthetic construct
agtgggtctcaaccactataaag

SEQ ID NO: 69 - DNA, 12 bp, Artificial Sequence
  Description of Artificial Sequence/Note = synthetic construct
cctctctgtgcc

SEQ ID NO: 70 - DNA, 21 bp, Artificial Sequence
  Description of Artificial Sequence/Note = synthetic construct
acgtcattgccctttctgtcc

SEQ ID NO: 71 - DNA, 23 bp, Artificial Sequence
  Description of Artificial Sequence/Note = synthetic construct
cacacagtcatggttccaacacg

SEQ ID NO: 72 - DNA, 23 bp, Artificial Sequence
  Description of Artificial Sequence/Note = synthetic construct
agtgggtctcaaccactataaat

SEQ ID NO: 73 - DNA, 19 bp, Artificial Sequence
  Description of Artificial Sequence/Note = synthetic construct
ggagaaaggccagtccatc

SEQ ID NO: 74 - DNA, 13 bp, Artificial Sequence
  Description of Artificial Sequence/Note = synthetic construct
gacgacatgatcc

SEQ ID NO: 75 - DNA, 18 bp, Artificial Sequence
  Description of Artificial Sequence/Note = synthetic construct
atttgacgtgtccaacgc

SEQ ID NO: 76 - DNA, 20 bp, Artificial Sequence
  Description of Artificial Sequence/Note = synthetic construct
tggaactctggttgaaactg

SEQ ID NO: 77 - DNA, 19 bp, Artificial Sequence
  Description of Artificial Sequence/Note = synthetic construct
ggagaaaggccagtccatt

SEQ ID NO: 78 - DNA, 24 bp, Artificial Sequence
  Description of Artificial Sequence/Note = synthetic construct
atctgattggctttctgaggttta

SEQ ID NO: 79 - DNA, 11 bp, Artificial Sequence
  Description of Artificial Sequence/Note = synthetic construct
gctgggtgggg

SEQ ID NO: 80 - DNA, 22 bp, Artificial Sequence
  Description of Artificial Sequence/Note = synthetic construct
ccactggctgctgttctgaaac

SEQ ID NO: 81 - DNA, 24 bp, Artificial Sequence
  Description of Artificial Sequence/Note = synthetic construct
aagcgaccatccccacatccattc

SEQ ID NO: 82 - DNA, 24 bp, Artificial Sequence
  Description of Artificial Sequence/Note = synthetic construct
atctgattggctttctgaggtttg

SEQ ID NO: 83 - DNA, 20 bp, Artificial Sequence
  Description of Artificial Sequence/Note = synthetic construct
ctcatttggccactctgcaa

SEQ ID NO: 84 - DNA, 13 bp, Artificial Sequence
  Description of Artificial Sequence/Note = synthetic construct
attggacttgccc

SEQ ID NO: 85 - DNA, 22 bp, Artificial Sequence
  Description of Artificial Sequence/Note = synthetic construct
ccactggctgctgttctgaaac

SEQ ID NO: 86 - DNA, 24 bp, Artificial Sequence
  Description of Artificial Sequence/Note = synthetic construct
aagcgaccatccccacatccattc

SEQ ID NO: 87 - DNA, 20 bp, Artificial Sequence
  Description of Artificial Sequence/Note = synthetic construct
ctcatttggccactctgcag

SEQ ID NO: 88 - DNA, 21 bp, Artificial Sequence
  Description of Artificial Sequence/Note = synthetic construct
cttatatagctgcgcgggaac

SEQ ID NO: 89 - DNA, 13 bp, Artificial Sequence
  Description of Artificial Sequence/Note = synthetic construct
aaggttgtcctgc

SEQ ID NO: 90 - DNA, 24 bp, Artificial Sequence
  Description of Artificial Sequence/Note = synthetic construct
aaatgagacggtttggggagcgag

SEQ ID NO: 91 - DNA, 24 bp, Artificial Sequence
  Description of Artificial Sequence/Note = synthetic construct
gtgacagagaatgagtttgcgatg

SEQ ID NO: 92 - DNA, 21 bp, Artificial Sequence
  Description of Artificial Sequence/Note = synthetic construct
cttatatagctgcgcgggaat

SEQ ID NO: 93 - DNA, 25 bp, Artificial Sequence
  Description of Artificial Sequence/Note = synthetic construct
aatcttacttatcgaaccggactta

SEQ ID NO: 94 - DNA, 15 bp, Artificial Sequence
  Description of Artificial Sequence/Note = synthetic construct
ttttgcttgttgccc

SEQ ID NO: 95 - DNA, 24 bp, Artificial Sequence
  Description of Artificial Sequence/Note = synthetic construct
aaatgagacggtttggggagcgag

SEQ ID NO: 96 - DNA, 24 bp, Artificial Sequence
  Description of Artificial Sequence/Note = synthetic construct
gtgacagagaatgagtttgcgatg

SEQ ID NO: 97 - DNA, 25 bp, Artificial Sequence
  Description of Artificial Sequence/Note = synthetic construct
aatcttacttatcgaaccggacttc

SEQ ID NO: 98 - DNA, 18 bp, Artificial Sequence
  Description of Artificial Sequence/Note = synthetic construct
catcctccagcgccctca

SEQ ID NO: 99 - DNA, 12 bp, Artificial Sequence
  Description of Artificial Sequence/Note = synthetic construct
gtcacagcactg

SEQ ID NO: 100 - DNA, 21 bp, Artificial Sequence
  Description of Artificial Sequence/Note = synthetic construct
atatttcacctggcctttgag

SEQ ID NO: 101 - DNA, 22 bp, Artificial Sequence
  Description of Artificial Sequence/Note = synthetic construct
tacagtctcatgaggatagccc

SEQ ID NO: 102 - DNA, 17 bp, Artificial Sequence
  Description of Artificial Sequence/Note = synthetic construct
atcctccagcgccctcg

SEQ ID NO: 103 - DNA, 22 bp, Artificial Sequence
  Description of Artificial Sequence/Note = synthetic construct
gatcacttttccacagctggac

SEQ ID NO: 104 - DNA, 13 bp, Artificial Sequence
  Description of Artificial Sequence/Note = synthetic construct
caccttgagaatg

SEQ ID NO: 105 - DNA, 22 bp, Artificial Sequence
  Description of Artificial Sequence/Note = synthetic construct
gctctaaagagaagctcacagc

SEQ ID NO: 106 - DNA, 22 bp, Artificial Sequence
  Description of Artificial Sequence/Note = synthetic construct
cacctgagattaaaaggtctgc

SEQ ID NO: 107 - DNA, 22 bp, Artificial Sequence
  Description of Artificial Sequence/Note = synthetic construct
gatcacttttccacagctggag

SEQ ID NO: 108 - DNA, 20 bp, Artificial Sequence
  Description of Artificial Sequence/Note = synthetic construct
atgcaggagaatgaccagcc

SEQ ID NO: 109 - DNA, 12 bp, Artificial Sequence
  Description of Artificial Sequence/Note = synthetic construct
gtcctgcacctg

SEQ ID NO: 110 - DNA, 22 bp, Artificial Sequence
  Description of Artificial Sequence/Note = synthetic construct
ctaaagacaagtctccagtggc

SEQ ID NO: 111 - DNA, 22 bp, Artificial Sequence
  Description of Artificial Sequence/Note = synthetic construct
gtcatgacagctacaggaaagg

SEQ ID NO: 112 - DNA, 21 bp, Artificial Sequence
  Description of Artificial Sequence/Note = synthetic construct
gatgcaggagaatgaccagct